(19) United States
(12) Patent Application Publication
(10) Pub. No.: US 2008/0070987 A1
(43) Pub. Date: Mar. 20, 2008

(54) META-XYLYLENEDIAMINE VANADATE SALTS

(76) Inventors: Francesc Yraola Font, Barcelona (ES); Silvia Garcia Vicente, Sant Feliu de Llobregat (ES); Juan Fernandez Recio, Barcelona (ES); Fernando Albericio Palomera, Barcelona (ES); Antonio Zorzano Olarte, Barcelona (ES); Miriam Royo Exposito, Barcelona (ES); Luc Marti Clauzel, Barcelona (ES)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/748,282

(22) Filed: May 14, 2007

Related U.S. Application Data
(60) Provisional application No. 60/800,057, filed on May 12, 2006.
(60) Provisional application No. 60/800,058, filed on May 12, 2006.

(51) Int. Cl. A61K 31/185; A61P 3/10; C07C 269/00

(52) U.S. Cl. 514/576; 560/115

(57) ABSTRACT

The invention provides compounds of Formula (I): 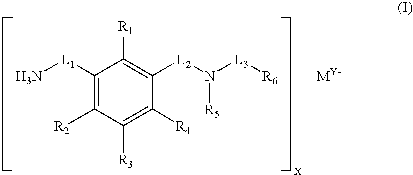
and Formula (II) 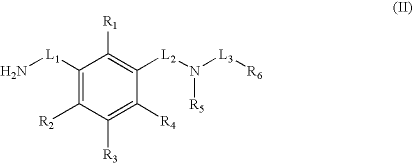 or a pharmaceutically-acceptable salt, solvate, or hydrate thereof. Compounds of this invention, or pharmaceutical compositions thereof, are useful for treating diabetes, elevated plasma glucose levels, and/or ketoacidosis in mammals.

This applications claims priority to U.S. provisional application Ser. No. 60/800,058, filed May 12, 2006, and U.S. provisional application Ser. No. 60/800,057, filed May 12, 2006, the disclosure of each of which are explicitly incorporated by reference herein.

### BACKGROUND

1. Field of the Invention

This invention relates to compounds of Formulae (I) and (II), pharmaceutically acceptable salt thereof, and pharmaceutical compositions thereof, useful for treating human type I and type II diabetes.

2. Background of the Related Art

Diabetes, especially in its most common form Diabetes mellitus, is a major global health problem that is recognized by the World Health Organization to be reaching epidemic proportions. It is now the fourth leading cause of death in most developed countries and a disease that is increasing rapidly in countries undergoing industrialization.

Diabetes mellitus is a metabolic disorder in which the ability to oxidize carbohydrates is practically lost, usually due to faulty pancreatic activity, especially of the islets of Langerhans, and consequent disturbance of normal insulin mechanism. It is characterized by abnormally elevated glucose levels in the plasma and urine, by excessive urine excretion and by episodic ketoacidosis. Additional symptoms of diabetes mellitus include excessive thirst, glucosuria, polyuria, lipidema and hunger. If left untreated the disease can lead to fatal ketoacidosis. Diabetes mellitus can eventually damage the eyes, kidneys, heart and limbs and can endanger pregnancy. Clinical criteria that establish an individual as suffering from diabetes mellitus include fasting plasma glucose levels in excess of 126 mg/dl (7 mmol/L; normal levels are typically less than 100 mg/dl (<5.6 mmol/L)). Alternatively, patients may show a plasma glucose levels in excess of 200 mg/dL (11 mmol/L) at two times points during a glucose tolerance test (GTT), one of which must be within 2 hrs of ingestion of glucose.

Diabetes mellitus is usually classified into two major types, type I diabetes and type II diabetes. Type I diabetes, or insulin-dependent Diabetes mellitus (IDDM), is defined by development of ketoacidosis in the absence of insulin therapy. Type I diabetes most often manifests in childhood and is therefore also called juvenile onset diabetes. Rapid in onset and progress, it accounts for about 10 to 15 percent of all cases. Type II diabetes, or non-insulin-dependent Diabetes mellitus (NIDDM), is characterized by persistent hyperglycemia but rarely by ketoacidosis. Type II diabetes typically manifests after age 40 and progresses slowly. Due to its late onset, it has formerly been called adult-onset diabetes. Type II diabetes, which is by far the most frequently occurring type of diabetes, is often not accompanied by clinical illness in its initial stages and is detected instead by elevated blood or urine glucose levels.

Two major forms of type II diabetes are to be distinguished in the basis of their association (or not) with obesity. Of the two, the form associated with obesity is of increasing importance. Type II diabetes associated with obesity is presently developing at an epidemic rate and is thus of major interest. For example, in the United States the proportion of the population under 40 that can be clinically defined as obese now exceeds 25%. Even many children are obese and are developing type II diabetes at an alarming rate.

Diabetes type I and 2 are both now considered as a group of disorders with multiple causes, rather than a single disorder. Common to diabetes type I and 2 is that entry of glucose into cells is impaired. Entry of glucose into cells is typically catalyzed by insulin, a hormone secreted by Langerhans cells in the pancreas. By facilitating entry of sugar glucose into tissue cells of the body insulin provides energy for metabolic activities. Impairment of glucose uptake may be a result either of a deficiency in the amount of insulin produced in the body or of altered target cells not enabling the cells to take up glucose. Impairment of glucose uptake results in excess glucose build-up in the blood and excreted in the urine.

Insulin elicits anabolic and anti-catabolic responses by activation of several intracellular signalling pathways. The actions of insulin are initiated by its binding to the insulin receptor, which leads to the activation of the receptor's intrinsic tyrosine kinase (Hubbard et al., 1994, Nature 372: 746-754; Hubbard, 1997, EMBO J. 16: 5572-5581). The function of the receptor tyrosine kinase is essential for the biological effects of insulin (Hubbard et al., 1994, Id.; Hubbard, 1997, Id.; Ebina et al., 1985, Cell 40: 747-758; Ullrich et al., 1985, Nature 313: 756-761; White & Kahn, 1994, J. Biol. Chem. 269: 1-4). Insulin receptors phosphorylate several immediate substrates including insulin receptor substrate (IRS) proteins (White & Kahn, 1994, Id.). These events lead to the activation of downstream signalling molecules such as phosphatidylinositol 3-kinase, protein kinase B or atypical forms of protein kinase C.

The etiology of type I diabetes almost always includes a severe or total reduction in insulin production. This reduction is typically the result of an autoimmune destruction of beta-cells in the pancreas that are responsible for producing insulin. The most common therapy for insulin dependent Diabetes mellitus (type I diabetes) is the provision of insulin by injection, thereby replacing the deficiency.

Type II diabetes can result from genetic defects that cause both insulin resistance and insulin deficiency. In type II diabetes, the pancreas often produces a considerable quantity of insulin, whereas the hormone is unable to promote the utilization of glucose by tissues. In fact, a hallmark of type II diabetes is insulin resistance. A subset of diabetic patients showed severe insulin resistance and they require more than 2 U of insulin per kg and day (Tritos & Mantzoros, 1998, J. Clin. Endocrinol. Metab. 83: 3025-3030; Vestergaard et al., 2001, J. Intern. Med. 250: 406-414. The molecular basis for insulin resistance in type II diabetes remains poorly understood, however. Several studies have shown that obesity or type II diabetes are characterized by modest decreases in insulin receptor number (Olefsky et al., 1985, Amer. J. Med. 79: 12-22), reduction in insulin-stimulated receptor tyrosine kinase activity and defects in receptor-mediated IRS phosphorylation or phosphatidylinositol 3-kinase or protein kinase C-□ activation (Olefsky et al., 1985, Id; Beeson et al., 2003, Diabetes 52: 1926-1934; Caro et al., 1987, J. Clin. Invest 79: 1330-1337; Goodyear et al., 1995, J. Clin. Invest 95: 2195-2204; Kim et al., 1999, J. Clin. Invest 104: 733-741). Thus, at least a subset of type II diabetic patients have clear defects in insulin signalling that could be overcome by treatment aimed at augmenting the insulin signalling cascade, inter alia, by providing an insulin replacement that bypasses the insulin receptor.

Various efforts have been made to treat diabetes and in particular insulin resistant diabetes type II. One such way of curing these conditions is to provide so called “insulin mimetics”, i.e. compounds capable of “mimicking” the functions of insulin such as to enable cells to take up glucose.

Several inorganic compounds have been reported to mimic the effects of insulin, in vivo as well as in isolated cells and tissues. Such mimetics include vanadium (IV)/(V) compounds. (Heyliger et al., 1985, Science 227: 1474-7); selenates (McNeill et al., 1991, Diabetes 40: 1675-8), lithium salts (Rodriquez-Gil et al., 1993, Arch. Biochem. Biophys. 301: 411-5), tungsten (VI) and molybdenum (VI) compounds (U.S. Pat. No. 5,595,763 and Li et al., 1995, Biochemistry 34: 6218-6225)).

Among the above inorganic compounds, vanadium and its derivatives have been proven as potent insulin-mimetics. There is convincing evidence for the effects of vanadates and peroxovanadium complexes (vanadium in its +5 oxidation state combined with oxygen, in particular orthovandate VO₄³⁻, see U.S. Pat. No. 4,882,171), and vanadyl VO²⁺ salts and complexes (vanadium in its +4 oxidation state; see U.S. Pat. No. 5,300,496) to increase cells' susceptibility for glucose uptake. Vanadium compounds are currently undergoing clinical trials in Europe and America. However, even though promising results for the transport of glucose into cells have been gathered, administration of vanadium compounds is accompanied by serious toxicity problems at effective doses. Administered concentrations must be close to toxic levels, if desired insulin-mimetic effects in animals are to be achieved. Considerable side effects are observed for vanadium-treatment that are independent from the chemical nature of the specific vanadium used for therapy (Domingo et al., 1991, Toxicology 66: 279-87.). Serious problems with vanadium compounds toxicity are observed at any kind of dosage suitable for lowering blood glucose levels, including a significant mortality rate.

Semicarbazide-sensitive amine oxidase (SSAO)/Vascular Adhesion Protein-1 (VAP-1) is a bifunctional membrane protein. One function of this protein is as a copper-containing ectoenzyme with amine oxidase activity that can be inhibited by carbonyl-reactive compounds such as semicarbazide (Lyles, 1996, Int. J. Biochem. Cell Biol. 28:259-274). SSAO oxidizes a primary amine into the corresponding aldehyde with production of hydrogen peroxide and ammonia according to the following reaction:
R—CH₂—NH₂+O₂→R—CHO+H₂O₂+NH₃

SSAO/VAP-1 is expressed in a variety of tissues, including endothelial cells, lung, smooth muscle cells, and (under normal conditions, highly expressed) in adipose tissue cells. SSAO/VAP-1 is not expressed in 3T3-L1 fibroblasts, but is induced during adipogenesis (Fontana et al., 2001, Biochem. J. 356:769-777; Moldes et al., 1999, J. Biol. Chem. 274:9515-9523). This suggests that SSAO/VAP-1 is a member of the adipogenic gene program and, in addition, that SSAO/VAP-1 may contribute to the acquisition of some final characteristics of fully differentiated adipose cells.

SSAO substrates are known to strongly stimulate glucose transport and recruitment of GLUT4 to the cell surface in isolated rat adipocytes or 3T3-L1 adipocytes (Enrique-Tarancon et al., 1998, J. Biol. Chem. 273:8025-8032; Enrique-Tarancon et al., 2000, Biochem. J. 350:171-180; Fontana et al., 2001, Biochem. J. 356:769-777; Marti et al., 1998, J Pharmacol. Exp. Ther. 285:342-349). Stimulation of glucose transport by SSAO substrates has also been demonstrated in isolated human adipocytes (Morin et al., 2001, J Pharmacol. Exp. Ther. 297:563-572).

Moreover, transport of glucose into cells in vivo has been mediated by using vanadate in combination with substrates of semicarbazide-sensitive amine-oxidase (SSAO), such as benzylamine or tyramine. As reported by Enrique-Tarancon et al. (1998, J. Biol. Chem. 273: 8025-8032 and Enrique-Tarancon et al., 2000, Biochem. J. 350: 171-180 or WO 02/38152), a combination of amines such as benzylamine or tyramine with vanadate was found to stimulate glucose transport and GLUT4 translocation in rat 3T3-L1 adipocytes. According to Enrique-Tarancon et al. (1998, 2000, supra) glucose transport is stimulated by an increase of GLUT4 carrier concentration on the cell surface, resulting from potent tyrosine phosphorylation. Similarly, Marti et al. (1998, J. Pharmacol. Exper. Therap. 285: 342-349) reported that glucose transport was stimulated using a combination of tyramine and vanadate. According to Marti et al. (1998, supra) stimulation of glucose transport was sensitive to MAO (monoamine oxidase) and SSAO inhibitors and to catalase. Marti et al. (1995, supra) also disclosed the use of vanadate in combination with tyramine. Patent application WO 02/38152 A1 describes a pharmaceutical combination formed by vanadium (IV)/(V) compounds and amines of the semicarbazide-sensitive amine oxidase substrates group, which is potently synergic in producing an insulin effect. More recently, in vivo studies have also demonstrated the anti-diabetic properties of the combination of benzylamine or other arylalkylamines with vanadium in experimental models of type I and type II diabetes. (Marti, et al. Diabetes. 2003, 50(9), 2061-8; Abella, et al., Diabetes 2003, 52:1004-1013) Thus, this combination is useful at low concentrations of the metal. However, these successes are tempered with the need to establish even the lowest possible effective doses for vanadate in order to avoid negative side effects of treatment due to toxicity of vanadate. Because the bifunctional protein of SSAO and vascular adhesion protein-1 (SSAO/VAP-1) is highly expressed in adipocytes, substrates that bind to SSAO are desirable.

In isolated rat adipocytes, the combination of substrates of SSAO with low ineffective vanadate concentrations produces a potent stimulation of glucose transport, which is abolished by semicarbazide and catalase. This combination also induces insulin-sensitive glucose transporter isoform 4 (GLUT4) recruitment to the cell surface, lipogenesis, and an inhibition of lipolysis. These observations indicate that the SSAO-dependent generation of hydrogen peroxide may be responsible for these effects via a chemical interaction with vanadate, which can form peroxovanadate, a known insulin-mimetic agent. Abella, et al., Diabetes. 2003, 52, 1004-1013.

Further, in vivo studies, known in the art, demonstrate the antidiabetic properties of the combination of benzylamine or other arylalkylamines with vanadium in experimental models of type I or type II diabetes.

Despite all the research efforts of the past, the treatment and/or prevention of Diabetes mellitus are far from being satisfactory. Therefore, it is useful to find new antidiabetic drugs, especially of the single ingredient kind. This is so because, in general the administration of a two-ingredient drug is less satisfactory than the administration of a single ingredient one, from the dosage and simplicity points of view.

For the subset of diabetic patients showing severe insulin resistance who require more than 2 U of insulin per kg and day, therapy with insulin replacement compounds that bypass the insulin receptor may be an efficient strategy. In addition, since patients with type I diabetes depend on parenteral exogenous insulin injections for metabolic control, the discovery of orally active compounds that mimic insulin's effects could lead to alternative therapies for this disorder.

Thus there is a need in the art for compounds and/or pharmaceutical compositions that mimic the effects of insulin, or preferably, are insulin replacement compounds that act, for example, in the insulin signalling cascade at a point downstream from the insulin receptor, thereby overcoming severe insulin resistance caused, inter alia, by diminution of insulin receptor molecules at the cell surface.

### SUMMARY OF THE INVENTION

In one aspect, the invention provides compounds of Formula (I): 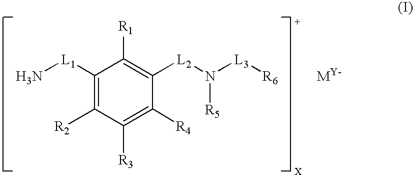
or a pharmaceutically-acceptable salt, solvate, or hydrate thereof, wherein

M is a negatively charged vanadium complex comprising vanadium V and oxygen, or vanadium, oxygen, and 1 or 2 hydroxy groups;

Y is an integer from 1 to 10;

X is an integer from 1 to 10;

L₁ and L₂ are independently (C₁-C₆)alkylene;

L₃ is —C(O)— or —S(O)₂—;

R₁, R₂, R₃, and R₄ are independently H, (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, or nitro;

R₅ is H or (C₁-C₆)alkyl;

R₆ is (C₁-C₆)alkoxy, (C₂-C₆)alkenyl, (C₂-C₆)alkenyloxy, (C₁-C₆)alkyl, (C₂-C₆)alkynyl, (C₂-C₆)alkynyloxy, aryl, (C₁-C₆)haloalkyl, (C₁-C₆)haloalkoxy, NR₇R₈, —CH(R₉)NR₁₀R₁₁, or —CH₂CH₂NR₁₀R₁₁, wherein the aryl is optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl;

R₇ and R₈ are independently H or (C₁-C₆)alkyl;

R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; and

R₁₀ and R₁₁ are independently H, (C₁-C₆)alkyl, (C₁-C₆)alkylcarbonyl, formyl, or (C₁-C₆)alkoxycarbonyl.

In another aspect, the invention provides a pharmaceutical composition comprising a compound according to Formula (I), or a pharmaceutically-acceptable solvate, hydrate, or salt thereof, and at least one pharmaceutically-acceptable excipient, diluent or adjuvant thereof.

In another aspect, the invention provides a method of treating diabetes in a mammal comprising administering to the mammal in need of such treatment a therapeutically effective amount of a compound of Formula (I) or a pharmaceutically-acceptable solvate, hydrate, or salt thereof.

In another aspect, the invention provides a method of treating a disease or disorder characterized by elevated glucose levels in the plasma in a mammal comprising administering to the mammal in need of such treatment a therapeutically effective amount of a compound of Formula (I) or a pharmaceutically-acceptable solvate, hydrate, or salt thereof.

In another aspect, the invention provides a method of treating ketoacidosis in a mammal comprising administering to the mammal in need of such treatment a therapeutically effective amount of a compound of Formula (I) or a pharmaceutically-acceptable solvate, hydrate, or salt thereof.

In another aspect, the invention provides a kit comprising a combination of a compound of Formula (I) and materials or other reagents useful in preparing or administering pharmaceutical compositions of said compounds. Solutions or diluents provided in the kits of the invention are preferably aqueous solutions or diluents.

Most preferably, the kit comprises the compounds of the invention in a single pharmaceutical composition in one or more containers. The container itself may be useful for administering the pharmaceutical compositions of the invention, inter alia, as an inhalant, syringe, pipette, eye dropper or other such apparatus, whereby the pharmaceutical composition of the invention can be administered for example by injection. The pharmaceutical compositions of the invention or components thereof can be provided in dried or lyophilized form, wherein reconstitution is provided by the addition of the appropriate solvent that is advantageously included in the kit. Instructions for preparing or reconstituting the pharmaceutical composition or administration thereof are also advantageously included.

In yet another aspect, the invention provides compounds of Formula (II):

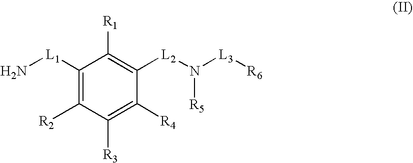

wherein
L₁ and L₂ are independently (C₁-C₆)alkylene;
L₃ is —C(O)— or —S(O)₂—;
R₁, R₂, R₃, and R₄ are independently H, (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, or nitro;
R₅ is H or (C₁-C₆)alkyl;
R₆ is (C₁-C₆)alkoxy, (C₂-C₆)alkenyl, (C₂-C₆)alkenyloxy, (C₁-C₆)alkyl, (C₂-C₆)alkynyl, (C₂-C₆)alkynyloxy, aryl, (C₁-C₆)haloalkyl, (C₁-C₆)haloalkoxy, NR₇R₈, —CH(R₉)NR₁₀R₁₁, or —CH₂CH₂NR₁₀R₁₁, wherein the aryl is optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl;
R₇ and R₈ are independently H or (C₁-C₆)alkyl;
R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; and
R₁₀ and R₁₁ are independently H, (C₁-C₆)alkyl, (C₁-C₆)alkylcarbonyl, formyl, or (C₁-C₆)alkoxycarbonyl; with the proviso that Formula (II) does not encompass N-(3-(aminomethyl)benzyl)acetamide.

Specific embodiments of the present invention will become evident from the following more detailed description of certain preferred embodiments and the claims.

### BRIEF DESCRIPTION OF THE DRAWINGS

FIG. 1 is a graphical illustration of the hexaquis(benzylammonium) decavanadate effects on glucose transport in isolated rat adipocytes. In this Figure, V corresponds to the rate of 2-deoxyglucose transport (expressed relative to the basal rate), and the results are mean +standard error of the mean (SEM). The adipocytes were incubated in the absence of stimulants in the following conditions: basal (1); in the presence of 100 nM insulin (2); in the presence of hexaquis(benzylammonium) decavanadate at concentrations of 0.5 μM (3), 1 μM (4), 2.5 μM (5), 5 μM (6), 10 μM (7), 25 μM (8), 50 μM (9), and 100 μM (10). The cells were also incubated in the presence of the semicarbazide inhibitor (1 mM), and 10 μM hexaquis(benzylammonium) decavanadate (11), 25 μM of hexaquis(benzylammonium) decavanadate (12) or 50 μM hexaquis(benzylammonium) decavanadate (13). In parallel, the cells were incubated in presence of 100 μM of sodium vanadate and 100 μM of benzylamine, in the absence (14) or in the presence of 1 mM of semicarbazide (15).

FIG. 2 is a graphical illustration of the effects of hexaquis(benzylammonium) decavanadate, pentaquis(benzylammonium) decavanadate and tetraquis(benzylammonium) decavanadate on glucose transport in isolated rat adipocytes. V corresponds to the rate of 2-deoxyglucose uptake (expressed as relation with basal group), and the results are mean +standard error mean. The adipocytes were incubated in the absence of stimulants in the following conditions: basal (1); in the presence of 100 nM of insulin (2); in the presence of hexaquis(benzylammonium) decavanadate at concentrations of 10 μM (3) and 25 μM (4), pentaquis(benzylammonium) decavanadate at concentrations of 10 μM (8) and 25 μM (9), and tetraquis(benzylammonium) decavanadate at concentrations of 10 μM (11) and 25 μM (12). The cells were also incubated in the presence of the semicarbazide inhibitor (1 mM) and, 25 μM of hexaquis(benzylammonium) decavanadate (5), 25 μM of pentaquis(benzylammonium) decavanadate (10) or 25 μM of tetraquis(benzylammonium) decavanadate (13). In parallel, the cells were incubated in the presence of 100 μM of sodium vanadate (6) or in the presence of 250 μM of sodium vanadate (7).

FIG. 3 is a graphical illustration of hexaquis(benzylammonium) decavanadate chronic treatment effect on glycemia of diabetic rats by streptozotocin. In FIG. 3, [G] corresponds to the blood concentration of glucose (expressed in mg/dl) measured at different days of treatment (t/d). Diabetic rats were treated, by mini-osmotic pumps, with buffered solution (black diamonds), with hexaquis(benzylammonium) decavanadate (2.5 μmol/kg/day) (black squares) or with identical dose of sodium decavanadate (white circles).

FIG. 4 is a graphical illustration of the chronic and oral treatment with hexaquis(benzylammonium) decavanadate on glycemia of diabetic rats by estreptozotocine. In FIG. 4, [G] corresponds to the blood concentration of glucose (expressed in mg/dl) measured at different days of treatment (t/d). Diabetic rats were treated with a single daily oral dose of hexaquis(benzylammonium) decavanadate (5 μmol/kg/day between day 0 and day 7 marked with an arrow, and 10 μmol/kg/day from 7 days of treatment) (black squares) or with identical dose of sodium decavanadate (black diamonds). Glycemia in non-diabetic rats is also represented in the figure (black triangles).

FIG. 5A through 5C show the stimulatory effects of hexaquis(benzylammonium) decavanadate (B6V10), pentaquis(benzyl ammonium) decavanadate (B5V10) and tetraquis (benzyl ammonium) decavanadate (B4V10) on glucose transport in adipose cells. All values shown are the mean ±SEM of 4-5 observations per group, and *, indicates a significant stimulation of 2-DG uptake compared with basal transport value at P<0.001. In FIG. 5A, †, indicates a significant stimulation of 2-DG uptake compared with basal transport value at P<0.05.

FIG. 6A shows chemical structures of advantageous embodiments of the arylalkylamine components of the insulin replacement compounds of the invention.

FIG. 6B shows the effects of vanadium salts of arylalkylamine components of the insulin replacement compounds of the invention on glucose transport by isolated rat adipocytes. *, indicates a significant stimulation of 2-DG uptake in groups incubated in the presence of 25 μM compounds compared with insulin-stimulated transport values at P<0.05.

FIG. 7A through 7E illustrate intracellular signalling pathway activated by hexaquis(benzylammonium) decavanadate in adipose cells and inhibited by phosphatidylinositol 3-kinase inhibitors (FIG. 7E). Values are mean ±SEM of 4-5 observations per group. *, indicates a significant stimulation of 2-DG uptake compared with basal transport value at P<0.05.

FIGS. 8A and 8B show the antidiabetic efficacy of administered hexaquis(benzylammonium) decavanadate in rat or mouse models of diabetes. All values are mean ±SEM of 6-7 observations. Two way ANOVA indicated the existence of significant differences between the B6V10 and the untreated or V10 groups (in FIG. 8A, P<0.01; FIG. 8B, P<0.001). Bonferroni post-tests for the results shown in FIG. 8A indicated significant differences in the B6V10 group compared to the untreated group from day 8 of treatment, at P<0.01.

FIGS. 9A and 9B illustrate results showing the antidiabetic efficacy of administered hexaquis(benzylammonium) decavanadate in streptozotocin-induced diabetic rat with undetectable circulating insulin. Values are mean ±SEM of 6-7 observations. Two way ANOVA indicated the existence of significant differences between the B6V10 and the untreated groups, at P<0.01 (FIG. 9A) or at P<0.05 (FIG. 9B).

### DETAILED DESCRIPTION OF PREFERRED EMBODIMENTS

The invention in one aspect provides compounds of Formula (I) wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and R₇ and R₈ are H.

In another aspect, the invention provides compounds of Formula (I) wherein M is V₁₀O₂₈; X is 6; Y is 6; L₁ is —CH₂—; L₂ is —CH₂—; L₃ is —C(O)—; R₁, R₂, R₃, R₄, and R₅ are H; and R₆ is (C₁-C₆)alkoxy.

In another aspect, the invention provides compounds of Formula (I) wherein M is V₁₀O₂₈; X is 6; Y is 6; L₁ is —CH₂—; L₂ is —CH₂—; L₃ is —C(O)—; R₁, R₂, R₃, R₄, and R₅ are H; and R₆ is (C₂-C₆)alkenyl.

In another aspect, the invention provides compounds of Formula (I) wherein M is V₁₀O₂₈; X is 6; Y is 6; L₁ is —CH₂—; L₂ is —CH₂—; L₃ is —C(O)—; R₁, R₂, R₃, R₄, and R₅ are H; and R₆ is (C₂-C₆)alkenyloxy.

In another aspect, the invention provides compounds of Formula (I) wherein M is V₁₀O₂₈; X is 6; Y is 6; L₁ is —CH₂—; L₂ is —CH₂—; L₃ is —C(O)—; R₁, R₂, R₃, R₄, and R₅ are H; and R₆ is (C₁-C₆)alkyl.

In another aspect, the invention provides compounds of Formula (I) wherein M is V₁₀O₂₈; X is 6; Y is 6; L₁ is —CH₂—; L₂ is —CH₂—; L₃ is —C(O)—; R₁, R₂, R₃, R₄, and R₅ are H; and R₆ is (C₂-C₆)alkynyl.

In another aspect, the invention provides compounds of Formula (I) wherein M is V₁₀O₂₈; X is 6; Y is 6; L₁ is —CH₂—; L₂ is —CH₂—; L₃ is —C(O)—; R₁, R₂, R₃, R₄, and R₅ are H; and R₆ is (C₂-C₆)alkynyloxy.

In another aspect, the invention provides compounds of Formula (I) wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is phenyl optionally substituted with 1 or 2 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; and R₇ and R₈ are independently H or (C₁-C₆)alkyl.

In another aspect, the invention provides compounds of Formula (I) wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is phenyl optionally substituted with 1 or 2 substituents independently selected from halogen, hydroxy, and NR₇R₈(C₁-C₆)alkyl; and R₇ and R₈ are independently H or (C₁-C₆)alkyl.

In another aspect, the invention provides compounds of Formula (I) wherein M is V₁₀O₂₈; X is 6; Y is 6; L₁ is —CH₂—; L₂ is —CH₂—; L₃ is —C(O)—; R₁, R₂, R₃, R₄, and R₅ are H; and R₆ is phenyl optionally substituted with 1 or 2 substituents independently selected from halogen, hydroxy, and —CH₂NH₂.

In another aspect, the invention provides compounds of Formula (I) wherein M is V₁₀O₂₈; X is 6; Y is 6; L₁ is —CH₂—; L₂ is —CH₂—; L₃ is —C(O)—; R₁, R₂, R₃, R₄, and R₅ are H; and R₆ is phenyl substituted with 1 substituent selected from halogen, hydroxy, and —CH₂NH₂.

In another aspect, the invention provides compounds of Formula (I) wherein M is V₁₀O₂₈; X is 6; Y is 6; L₁ is —CH₂—; L₂ is —CH₂—; L₃ is —C(O)—; R₁, R₂, R₃, R₄, and R₅ are H; and R₆ is (C₁-C₆)haloalkyl.

In another aspect, the invention provides compounds of Formula (I) wherein M is V₁₀O₂₈; X is 6; Y is 6; L₁ is —CH₂—; L₂ is —CH₂—; L₃ is —C(O)—; R₁, R₂, R₃, R₄, and R₅ are H; and R₆ is (C₁-C₆)haloalkoxy.

In another aspect, the invention provides compounds of Formula (I) wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is —NR₇R₈; and R₇ and R₈ are independently H or (C₁-C₆)alkyl.

In another aspect, the invention provides compounds of Formula (I) wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is —CH₂CH₂NR₁₀R₁₁ and R₁₀ and R₁₁ are independently H or (C₁-C₆)alkyl.

In another aspect, the invention provides compounds of Formula (I) wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is —CH₂CH₂NR₁₀R₁₁ and R₁₀ is H; and R₁₁ is (C₁-C₆)alkylcarbonyl.

In another aspect, the invention provides compounds of Formula (I) wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; and R₁₀ and R₁₁ are independently H, (C₁-C₆)alkyl, (C₁-C₆)alkylcarbonyl, formyl, or (C₁-C₆)alkoxycarbonyl.

In another aspect, the invention provides compounds of Formula (I) wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl optionally substituted with 1 or 2 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; R₁₀ is —H; and R₁₁ is (C₁-C₆)alkylcarbonyl.

In another aspect, the invention provides compounds of Formula (I) wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl; R₁₀ is —H; and R₁₁ is (C₁-C₆)alkylcarbonyl.

In another aspect, the invention provides compounds of Formula (I) wherein M is V₁₀O₂₈; X is 6; Y is 6; L₁ is —CH₂—; L₂ is —CH₂—; L₃ is —S(O)₂—; R₁, R₂, R₃, R₄, and R₅ are H; and R₆ is (C₁-C₆)alkoxy.

In another aspect, the invention provides compounds of Formula (I) wherein M is V₁₀O₂₈; X is 6; Y is 6; L₁ is —CH₂—; L₂ is —CH₂—; L₃ is —S(O)₂—; R₁, R₂, R₃, R₄, and R₅ are H; and R₆ is (C₂-C₆)alkenyl.

In another aspect, the invention provides compounds of Formula (I) wherein M is V₁₀O₂₈; X is 6; Y is 6; L₁ is —CH₂—; L₂ is —CH₂—; L₃ is —S(O)₂—; R₁, R₂, R₃, R₄, and R₅ are H; and R₆ is (C₂-C₆)alkenyloxy.

In another aspect, the invention provides compounds of Formula (I) wherein M is V₁₀O₂₈; X is 6; Y is 6; L₁ is —CH₂—; L₂ is —CH₂—; L₃ is —S(O)₂—; R₁, R₂, R₃, R₄, and R₅ are H; and R₆ is (C₁-C₆)alkyl.

In another aspect, the invention provides compounds of Formula (I) wherein M is V₁₀O₂₈; X is 6; Y is 6; L₁ is —CH₂—; L₂ is —CH₂—; L₃ is —S(O)₂—; R₁, R₂, R₃, R₄, and R₅ are H; and R₆ is (C₂-C₆)alkynyl.

In another aspect, the invention provides compounds of Formula (I) wherein M is V₁₀O₂₈; X is 6; Y is 6; L₁ is —CH₂—; L₂ is —CH₂—; L₃ is —S(O)₂—; R₁, R₂, R₃, R₄, and R₅ are H; and R₆ is (C₂-C₆)alkynyloxy.

In another aspect, the invention provides compounds of Formula (I) wherein M is V₁₀O₂₈; X is 6; Y is 6; L₁ is —CH₂—; L₂ is —CH₂—; L₃ is —S(O)₂—; R₁, R₂, R₃, R₄, and R₅ are H; R₆ is phenyl optionally substituted with 1 or 2 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; and R₇ and R₈ are independently H or (C₁-C₆)alkyl.

In another aspect, the invention provides compounds of Formula (I) wherein M is V₁₀O₂₈; X is 6; Y is 6; L₁ is —CH₂—; L₂ is —CH₂—; L₃ is —S(O)₂—; R₁, R₂, R₃, R₄, and R₅ are H; R₆ is phenyl optionally substituted with 1 substituent selected from halogen, hydroxy, and NR₇R₈(C₁-C₆)alkyl; and R₇ and R₈ are independently H or (C₁-C₆)alkyl.

In another aspect, the invention provides compounds of Formula (I) wherein M is V₁₀O₂₈; X is 6; Y is 6; L₁ is —CH₂—; L₂ is —CH₂—; L₃ is —S(O)₂—; R₁, R₂, R₃, R₄, and R₅ are H; and R₆ is phenyl optionally substituted with 1 substituent selected from halogen, hydroxy, and —CH₂NH₂.

In another aspect, the invention provides compounds of Formula (I) wherein M is V₁₀O₂₈; X is 6; Y is 6; L₁ is —CH₂—; L₂ is —CH₂—; L₃ is —S(O)₂—; R₁, R₂, R₃, R₄, and R₅ are H; and R₆ is phenyl substituted with 1 substituent selected from halogen, hydroxy, and —CH₂NH₂.

In another aspect, the invention provides compounds of Formula (I) wherein M is V₁₀O₂₈; X is 6; Y is 6; L₁ is —CH₂—; L₂ is —CH₂—; L₃ is —S(O)₂—; R₁, R₂, R₃, R₄, and R₅ are H; R₆ is phenyl optionally substituted with (C₁-C₆)alkyl.

In another aspect, the invention provides compounds of Formula (I) wherein M is V₁₀O₂₈; X is 6; Y is 6; L₁ is —CH₂—; L₂ is —CH₂—; L₃ is —S(O)₂—; R₁, R₂, R₃, R₄, and R₅ are H; and R₆ is (C₁-C₆)haloalkyl.

In another aspect, the invention provides compounds of Formula (I) wherein M is V₁₀O₂₈; X is 6; Y is 6; L₁ is —CH₂—; L₂ is —CH₂—; L₃ is —S(O)₂—; R₁, R₂, R₃, R₄, and R₅ are H; and R₆ is (C₁-C₆)haloalkoxy.

In another aspect, the invention provides compounds of Formula (I) wherein M is V₁₀O₂₈; X is 6; Y is 6; L₁ is —CH₂—; L₂ is —CH₂—; L₃ is —S(O)₂—; R₁, R₂, R₃, R₄, and R₅ are H; R₆ is NR₇R₈; and R₇ and R₈ are independently H or (C₁-C₆)alkyl.

In another aspect, the invention provides a pharmaceutical composition comprising a compound according to Formula (I), wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and R₇ and R₈ are H, or a pharmaceutically-acceptable solvate, hydrate, or salt thereof, and at least one pharmaceutically-acceptable excipient, diluent or adjuvant thereof.

In another aspect, the invention provides a pharmaceutical composition comprising a compound according to Formula (I), wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; and R₁₀ and R₁₁ are independently H, (C₁-C₆)alkyl, (C₁-C₆)alkylcarbonyl, formyl, or (C₁-C₆)alkoxycarbonyl, or a pharmaceutically-acceptable solvate, hydrate, or salt thereof, and at least one pharmaceutically-acceptable excipient, diluent or adjuvant thereof.

In another aspect, the invention provides a pharmaceutical composition comprising a compound according to Formula (I), wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl; R₁₀ is —H; and R₁₁ is (C₁-C₆)alkylcarbonyl, or a pharmaceutically-acceptable solvate, hydrate, or salt thereof, and at least one pharmaceutically-acceptable excipient, diluent or adjuvant thereof.

In another aspect, the invention provides a pharmaceutical composition comprising a compound according to Formula (I), wherein M is V₁₀O₂₈; X is 6; Y is 6; L₁ is —CH₂—; L₂ is —CH₂—; L₃ is —S(O)₂—; R₁, R₂, R₃, R₄, and R₅ are H; R₆ is phenyl optionally substituted with (C₁-C₆)alkyl, or a pharmaceutically-acceptable solvate, hydrate, or salt thereof, and at least one pharmaceutically-acceptable excipient, diluent or adjuvant thereof.

In another aspect, the invention provides a method of treating type I diabetes in a human comprising administering to the human in need of such treatment a therapeutically effective amount of a compound of Formula (I) or a pharmaceutically-acceptable salt, solvate, or hydrate thereof.

In another aspect, the invention provides a method of treating type I diabetes in a human comprising administering to the human in need of such treatment a therapeutically effective amount of a compound of Formula (I), or a pharmaceutically-acceptable salt, solvate, or hydrate thereof, wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and R₇ and R₈ are H.

In another aspect, the invention provides a method of treating type I diabetes in a human comprising administering to the human in need of such treatment a therapeutically effective amount of a compound of Formula (I), or a pharmaceutically-acceptable salt, solvate, or hydrate thereof, wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; and R₁₀ and R₁₁ are independently H, (C₁-C₆)alkyl, (C₁-C₆)alkylcarbonyl, formyl, or (C₁-C₆)alkoxycarbonyl.

In another aspect, the invention provides a method of treating type I diabetes in a human comprising administering to the human in need of such treatment a therapeutically effective amount of a compound of Formula (I), or a pharmaceutically-acceptable salt, solvate, or hydrate thereof, wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl; R₁₀ is —H; and R₁₁ is (C₁-C₆)alkylcarbonyl.

In another aspect, the invention provides a method of treating type I diabetes in a human comprising administering to the human in need of such treatment a therapeutically effective amount of a compound of Formula (I), or a pharmaceutically-acceptable salt, solvate, or hydrate thereof, wherein M is V₁₀O₂₈; X is 6; Y is 6; L₁ is —CH₂—; L₂ is —CH₂—; L₃ is —S(O)₂—; R₁, R₂, R₃, R₄, and R₅ are H; R₆ is phenyl optionally substituted with (C₁-C₆)alkyl.

In another aspect, the invention provides a method of treating type II diabetes in a human comprising administering to the human in need of such treatment a therapeutically effective amount of a compound of Formula (I) or a pharmaceutically-acceptable salt, solvate, or hydrate thereof.

In another aspect, the invention provides a method of treating type II diabetes in a human comprising administering to the human in need of such treatment a therapeutically effective amount of a compound of Formula (I), or a pharmaceutically-acceptable salt, solvate, or hydrate thereof, wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and R₇ and R₈ are H.

In another aspect, the invention provides a method of treating type II diabetes in a human comprising administering to the human in need of such treatment a therapeutically effective amount of a compound of Formula (I), or a pharmaceutically-acceptable salt, solvate, or hydrate thereof, wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; and R₁₀ and R₁₁ are independently H, (C₁-C₆)alkyl, (C₁-C₆)alkylcarbonyl, formyl, or (C₁-C₆)alkoxycarbonyl.

In another aspect, the invention provides a method of treating type II diabetes in a human comprising administering to the human in need of such treatment a therapeutically effective amount of a compound of Formula (I), or a pharmaceutically-acceptable salt, solvate, or hydrate thereof, wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl; R₁₀ is —H; and R₁₁ is (C₁-C₆)alkylcarbonyl.

In another aspect, the invention provides a method of treating type II diabetes in a human comprising administering to the human in need of such treatment a therapeutically effective amount of a compound of Formula (I), or a pharmaceutically-acceptable salt, solvate, or hydrate thereof, wherein M is V₁₀O₂₈; X is 6; Y is 6; L₁ is —CH₂—; L₂ is —CH₂—; L₃ is —S(O)₂—; R₁, R₂, R₃, R₄, and R₅ are H; R₆ is phenyl optionally substituted with (C₁-C₆)alkyl.

In another aspect, the invention provides a method of treating elevated plasma glucose levels in a human comprising administering to the human in need of such treatment a therapeutically effective amount of a compound of Formula (I) or a pharmaceutically-acceptable salt, solvate, or hydrate thereof.

In another aspect, the invention provides a method of treating elevated plasma glucose levels in a human comprising administering to the human in need of such treatment a therapeutically effective amount of a compound of Formula (I), or a pharmaceutically-acceptable salt, solvate, or hydrate thereof, wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and R₇ and R₈ are H.

In another aspect, the invention provides a method of treating elevated plasma glucose levels in a human comprising administering to the human in need of such treatment a therapeutically effective amount of a compound of Formula (I), or a pharmaceutically-acceptable salt, solvate, or hydrate thereof, wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; and R₁₀ and R₁₁ are independently H, (C₁-C₆)alkyl, (C₁-C₆)alkylcarbonyl, formyl, or (C₁-C₆)alkoxycarbonyl.

In another aspect, the invention provides a method of treating elevated plasma glucose levels in a human comprising administering to the human in need of such treatment a therapeutically effective amount of a compound of Formula (I), or a pharmaceutically-acceptable salt, solvate, or hydrate thereof, wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl; R₁₀ is —H; and R₁₁ is (C₁-C₆)alkylcarbonyl.

In another aspect, the invention provides a method of treating elevated plasma glucose levels in a human comprising administering to the human in need of such treatment a therapeutically effective amount of a compound of Formula (I), or a pharmaceutically-acceptable salt, solvate, or hydrate thereof, wherein M is V₁₀O₂₈; X is 6; Y is 6; L₁ is —CH₂—; L₂ is —CH₂—; L₃ is —S(O)₂—; R₁, R₂, R₃, R₄, and R₅ are H; R₆ is phenyl optionally substituted with (C₁-C₆)alkyl.

In another aspect, the invention provides a method of treating ketoacidosis in a human comprising administering to the human in need of such treatment a therapeutically effective amount of a compound of Formula (I) or a pharmaceutically-acceptable salt, solvate, or hydrate thereof.

In another aspect, the invention provides a method of treating ketoacidosis in a human comprising administering to the human in need of such treatment a therapeutically effective amount of a compound of Formula (I), or a pharmaceutically-acceptable salt, solvate, or hydrate thereof, wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and R₇ and R₈ are H.

In another aspect, the invention provides a method of treating ketoacidosis in a human comprising administering to the human in need of such treatment a therapeutically effective amount of a compound of Formula (I), or a pharmaceutically-acceptable salt, solvate, or hydrate thereof, wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; and R₁₀ and R₁₁ are independently H, (C₁-C₆)alkyl, (C₁-C₆)alkylcarbonyl, formyl, or (C₁-C₆)alkoxycarbonyl.

In another aspect, the invention provides a method of treating ketoacidosis in a human comprising administering to the human in need of such treatment a therapeutically effective amount of a compound of Formula (I), or a pharmaceutically-acceptable salt, solvate, or hydrate thereof, wherein M is V₁₀O₂₈; X is 6; Y is 6; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl; R₁₀ is —H; and R₁₁ is (C₁-C₆)alkylcarbonyl.

In another aspect, the invention provides a method of treating ketoacidosis in a human comprising administering to the human in need of such treatment a therapeutically effective amount of a compound of Formula (I), or a pharmaceutically-acceptable salt, solvate, or hydrate thereof, wherein M is V₁₀O₂₈; X is 6; Y is 6; L₁ is —CH₂—; L₂ is —CH₂—; L₃ is —S(O)₂—; R₁, R₂, R₃, R₄, and R₅ are H; R₆ is phenyl optionally substituted with (C₁-C₆)alkyl.

In another aspect, the invention provides compounds of Formula (I) wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and R₇ and R₈ are H.

In another aspect, the invention provides compounds of Formula (I) wherein M is V₁₀O₂₇OH; X is 5; Y is 5; L₁ is —CH₂—; L₂ is —CH₂—; L₃ is —C(O)—; R₁, R₂, R₃, R₄, and R₅ are H; and R₆ is (C₁-C₆)alkoxy.

In another aspect, the invention provides compounds of Formula (I) wherein M is V₁₀O₂₇OH; X is 5; Y is 5; L₁ is —CH₂—; L₂ is —CH₂—; L₃ is —C(O)—; R₁, R₂, R₃, R₄, and R₅ are H; and R₆ is (C₂-C₆)alkenyl.

In another aspect, the invention provides compounds of Formula (I) wherein M is V₁₀O₂₇OH; X is 5; Y is 5; L₁ is —CH₂—; L₂ is —CH₂—; L₃ is —C(O)—; R₁, R₂, R₃, R₄, and R₅ are H; and R₆ is (C₂-C₆)alkenyloxy.

In another aspect, the invention provides compounds of Formula (I) wherein M is V₁₀O₂₇OH; X is 5; Y is 5; L₁ is —CH₂—; L₂ is —CH₂—; L₃ is —C(O)—; R₁, R₂, R₃, R₄, and R₅ are H; and R₆ is (C₁-C₆)alkyl.

In another aspect, the invention provides compounds of Formula (I) wherein M is V₁₀O₂₇OH; X is 5; Y is 5; L₁ is —CH₂—; L₂ is —CH₂—; L₃ is —C(O)—; R₁, R₂, R₃, R₄, and R₅ are H; and R₆ is (C₂-C₆)alkynyl.

In another aspect, the invention provides compounds of Formula (I) wherein M is V₁₀O₂₇OH; X is 5; Y is 5; L₁ is —CH₂—; L₂ is —CH₂—; L₃ is —C(O)—; R₁, R₂, R₃, R₄, and R₅ are H; and R₆ is (C₂-C₆)alkynyloxy.

In another aspect, the invention provides compounds of Formula (I) wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is phenyl optionally substituted with 1 or 2 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; and R₇ and R₈ are independently H or (C₁-C₆)alkyl.

In another aspect, the invention provides compounds of Formula (I) wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is phenyl optionally substituted with 1 or 2 substituents independently selected from halogen, hydroxy, and NR₇R₈(C₁-C₆)alkyl; and R₇ and R₈ are independently H or (C₁-C₆)alkyl.

In another aspect, the invention provides compounds of Formula (I) wherein M is V₁₀O₂₇OH; X is 5; Y is 5; L₁ is —CH₂—; L₂ is —CH₂—; L₃ is —C(O)—; R₁, R₂, R₃, R₄, and R₅ are H; and R₆ is phenyl optionally substituted with 1 or 2 substituents independently selected from halogen, hydroxy, and —CH₂NH₂.

In another aspect, the invention provides compounds of Formula (I) wherein M is V₁₀O₂₇OH; X is 5; Y is 5; L₁ is —CH₂—; L₂ is —CH₂—; L₃ is —C(O)—; R₁, R₂, R₃, R₄, and R₅ are H; and R₆ is phenyl substituted with 1 substituent selected from halogen, hydroxy, and —CH₂NH₂.

In another aspect, the invention provides compounds of Formula (I) wherein M is V₁₀O₂₇OH; X is 5; Y is 5; L₁ is —CH₂—; L₂ is —CH₂—; L₃ is —C(O)—; R₁, R₂, R₃, R₄, and R₅ are H; and R₆ is (C₁-C₆)haloalkyl.

In another aspect, the invention provides compounds of Formula (I) wherein M is V₁₀O₂₇OH; X is 5; Y is 5; L₁ is —CH₂—; L₂ is —CH₂—; L₃ is —C(O)—; R₁, R₂, R₃, R₄, and R₅ are H; and R₆ is (C₁-C₆)haloalkoxy.

In another aspect, the invention provides compounds of Formula (I) wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is —NR₇R₈; and R₇ and R₈ are independently H or (C₁-C₆)alkyl.

In another aspect, the invention provides compounds of Formula (I) wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is —CH₂CH₂NR₁₀R₁₁ and R₁₀ and R₁₁ are independently H or (C₁-C₆)alkyl.

In another aspect, the invention provides compounds of Formula (I) wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is —CH₂CH₂NR₁₀R₁₁ and R₁₀ is H; and R₁₁ is (C₁-C₆)alkylcarbonyl.

In another aspect, the invention provides compounds of Formula (I) wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; and R₁₀ and R₁₁ are independently H, (C₁-C₆)alkyl, (C₁-C₆)alkylcarbonyl, formyl, or (C₁-C₆)alkoxycarbonyl.

In another aspect, the invention provides compounds of Formula (I) wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl optionally substituted with 1 or 2 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; R₁₀ is —H; and R₁₁ is (C₁-C₆)alkylcarbonyl.

In another aspect, the invention provides compounds of Formula (I) wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl; R₁₀ is —H; and R₁₁ is (C₁-C₆)alkylcarbonyl.

In another aspect, the invention provides compounds of Formula (I) wherein M is V₁₀O₂₇OH; X is 5; Y is 5; L₁ is —CH₂—; L₂ is —CH₂—; L₃ is —S(O)₂—; R₁, R₂, R₃, R₄, and R₅ are H; and R₆ is (C₁-C₆)alkoxy.

In another aspect, the invention provides compounds of Formula (I) wherein M is V₁₀O₂₇OH; X is 5; Y is 5; L₁ is —CH₂—; L₂ is —CH₂—; L₃ is —S(O)₂—; R₁, R₂, R₃, R₄, and R₅ are H; and R₆ is (C₂-C₆)alkenyl.

In another aspect, the invention provides compounds of Formula (I) wherein M is V₁₀O₂₇OH; X is 5; Y is 5; L₁ is —CH₂—; L₂ is —CH₂—; L₃ is —S(O)₂—; R₁, R₂, R₃, R₄, and R₅ are H; and R₆ is (C₂-C₆)alkenyloxy.

In another aspect, the invention provides compounds of Formula (I) wherein M is V₁₀O₂₇OH; X is 5; Y is 5; L₁ is —CH₂—; L₂ is —CH₂—; L₃ is —S(O)₂—; R₁, R₂, R₃, R₄, and R₅ are H; and R₆ is (C₁-C₆)alkyl.

In another aspect, the invention provides compounds of Formula (I) wherein M is V₁₀O₂₇OH; X is 5; Y is 5; L₁ is —CH₂—; L₂ is —CH₂—; L₃ is —S(O)₂—; R₁, R₂, R₃, R₄, and R₅ are H; and R₆ is (C₂-C₆)alkynyl.

In another aspect, the invention provides compounds of Formula (I) wherein M is V₁₀O₂₇OH; X is 5; Y is 5; L₁ is —CH₂—; L₂ is —CH₂—; L₃ is —S(O)₂—; R₁, R₂, R₃, R₄, and R₅ are H; and R₆ is (C₂-C₆)alkynyloxy.

In another aspect, the invention provides compounds of Formula (I) wherein M is V₁₀O₂₇OH; X is 5; Y is 5; L₁ is —CH₂—; L₂ is —CH₂—; L₃ is —S(O)₂—; R₁, R₂, R₃, R₄, and R₅ are H; R₆ is phenyl optionally substituted with 1 or 2 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; and R₇ and R₈ are independently H or (C₁-C₆)alkyl.

In another aspect, the invention provides compounds of Formula (I) wherein M is V₁₀O₂₇OH; X is 5; Y is 5; L₁ is —CH₂—; L₂ is —CH₂—; L₃ is —S(O)₂—; R₁, R₂, R₃, R₄, and R₅ are H; R₆ is phenyl optionally substituted with 1 substituent selected from halogen, hydroxy, and NR₇R₈(C₁-C₆)alkyl; and R₇ and R₈ are independently H or (C₁-C₆)alkyl.

In another aspect, the invention provides compounds of Formula (I) wherein M is V₁₀O₂₇OH; X is 5; Y is 5; L₁ is —CH₂—; L₂ is —CH₂—; L₃ is —S(O)₂—; R₁, R₂, R₃, R₄, and R₅ are H; and R₆ is phenyl optionally substituted with 1 substituent selected from halogen, hydroxy, and —CH₂NH₂.

In another aspect, the invention provides compounds of Formula (I) wherein M is V₁₀O₂₇OH; X is 5; Y is 5; L₁ is —CH₂—; L₂ is —CH₂—; L₃ is —S(O)₂—; R₁, R₂, R₃, R₄, and R₅ are H; and R₆ is phenyl substituted with 1 substituent selected from halogen, hydroxy, and —CH₂NH₂.

In another aspect, the invention provides compounds of Formula (I) wherein M is V₁₀O₂₇OH; X is 5; Y is 5; L₁ is —CH₂—; L₂ is —CH₂—; L₃ is —S(O)₂—; R₁, R₂, R₃, R₄, and R₅ are H; R₆ is phenyl optionally substituted with (C₁-C₆)alkyl.

In another aspect, the invention provides compounds of Formula (I) wherein M is V₁₀O₂₇OH; X is 5; Y is 5; L₁ is —CH₂—; L₂ is —CH₂—; L₃ is —S(O)₂—; R₁, R₂, R₃, R₄, and R₅ are H; and R₆ is (C₁-C₆)haloalkyl.

In another aspect, the invention provides compounds of Formula (I) wherein M is V₁₀O₂₇OH; X is 5; Y is 5; L₁ is —CH₂—; L₂ is —CH₂—; L₃ is —S(O)₂—; R₁, R₂, R₃, R₄, and R₅ are H; and R₆ is (C₁-C₆)haloalkoxy.

In another aspect, the invention provides compounds of Formula (I) wherein M is V₁₀O₂₇OH; X is 5; Y is 5; L₁ is —CH₂—; L₂ is —CH₂—; L₃ is —S(O)₂—; R₁, R₂, R₃, R₄, and R₅ are H; R₆ is NR₇R₈; and R₇ and R₈ are independently H or (C₁-C₆)alkyl.

In another aspect, the invention provides a pharmaceutical composition comprising a compound according to Formula (I), wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and R₇ and R₈ are H, or a pharmaceutically-acceptable solvate, hydrate, or salt thereof, and at least one pharmaceutically-acceptable excipient, diluent or adjuvant thereof.

In another aspect, the invention provides a pharmaceutical composition comprising a compound according to Formula (I), wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; and R₁₀ and R₁₁ are independently H, (C₁-C₆)alkyl, (C₁-C₆)alkylcarbonyl, formyl, or (C₁-C₆)alkoxycarbonyl, or a pharmaceutically-acceptable solvate, hydrate, or salt thereof, and at least one pharmaceutically-acceptable excipient, diluent or adjuvant thereof.

In another aspect, the invention provides a pharmaceutical composition comprising a compound according to Formula (I), wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl; R₁₀ is —H; and R₁₁ is (C₁-C₆)alkylcarbonyl, or a pharmaceutically-acceptable solvate, hydrate, or salt thereof, and at least one pharmaceutically-acceptable excipient, diluent or adjuvant thereof.

In another aspect, the invention provides a pharmaceutical composition comprising a compound according to Formula (I), wherein M is V₁₀O₂₇OH; X is 5; Y is 5; L₁ is —CH₂—; L₂ is —CH₂—; L₃ is —S(O)₂—; R₁, R₂, R₃, R₄, and R₅ are H; R₆ is phenyl optionally substituted with (C₁-C₆)alkyl, or a pharmaceutically-acceptable solvate, hydrate, or salt thereof, and at least one pharmaceutically-acceptable excipient, diluent or adjuvant thereof.

In another aspect, the invention provides a method of treating type I diabetes in a human comprising administering to the human in need of such treatment a therapeutically effective amount of a compound of Formula (I), or a pharmaceutically-acceptable salt, solvate, or hydrate thereof, wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and R₇ and R₈ are H.

In another aspect, the invention provides a method of treating type I diabetes in a human comprising administering to the human in need of such treatment a therapeutically effective amount of a compound of Formula (I), or a pharmaceutically-acceptable salt, solvate, or hydrate thereof, wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; and R₁₀ and R₁₁ are independently H, (C₁-C₆)alkyl, (C₁-C₆)alkylcarbonyl, formyl, or (C₁-C₆)alkoxycarbonyl.

In another aspect, the invention provides a method of treating type I diabetes in a human comprising administering to the human in need of such treatment a therapeutically effective amount of a compound of Formula (I), or a pharmaceutically-acceptable salt, solvate, or hydrate thereof, wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl; R₁₀ is —H; and R₁₁ is (C₁-C₆)alkylcarbonyl.

In another aspect, the invention provides a method of treating type I diabetes in a human comprising administering to the human in need of such treatment a therapeutically effective amount of a compound of Formula (I), or a pharmaceutically-acceptable salt, solvate, or hydrate thereof, wherein M is V₁₀O₂₇OH; X is 5; Y is 5; L₁ is —CH₂—; L₂ is —CH₂—; L₃ is —S(O)₂—; R₁, R₂, R₃, R₄, and R₅ are H; R₆ is phenyl optionally substituted with (C₁-C₆)alkyl.

In another aspect, the invention provides a method of treating type II diabetes in a human comprising administering to the human in need of such treatment a therapeutically effective amount of a compound of Formula (I), or a pharmaceutically-acceptable salt, solvate, or hydrate thereof, wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and R₇ and R₈ are H.

In another aspect, the invention provides a method of treating type II diabetes in a human comprising administering to the human in need of such treatment a therapeutically effective amount of a compound of Formula (I), or a pharmaceutically-acceptable salt, solvate, or hydrate thereof, wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; and R₁₀ and R₁₁ are independently H, (C₁-C₆)alkyl, (C₁-C₆)alkylcarbonyl, formyl, or (C₁-C₆)alkoxycarbonyl.

In another aspect, the invention provides a method of treating type II diabetes in a human comprising administering to the human in need of such treatment a therapeutically effective amount of a compound of Formula (I), or a pharmaceutically-acceptable salt, solvate, or hydrate thereof, wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl; R₁₀ is —H; and R₁₁ is (C₁-C₆)alkylcarbonyl.

In another aspect, the invention provides a method of treating type II diabetes in a human comprising administering to the human in need of such treatment a therapeutically effective amount of a compound of Formula (I), or a pharmaceutically-acceptable salt, solvate, or hydrate thereof, wherein M is V₁₀O₂₇OH; X is 5; Y is 5; L₁ is —CH₂—; L₂ is —CH₂—; L₃ is —S(O)₂—; R₁, R₂, R₃, R₄, and R₅ are H; R₆ is phenyl optionally substituted with (C₁-C₆)alkyl.

In another aspect, the invention provides a method of treating elevated plasma glucose levels in a human comprising administering to the human in need of such treatment a therapeutically effective amount of a compound of Formula (I), or a pharmaceutically-acceptable salt, solvate, or hydrate thereof, wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and R₇ and R₈ are H.

In another aspect, the invention provides a method of treating elevated plasma glucose levels in a human comprising administering to the human in need of such treatment a therapeutically effective amount of a compound of Formula (I), or a pharmaceutically-acceptable salt, solvate, or hydrate thereof, wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; and R₁₀ and R₁₁ are independently H, (C₁-C₆)alkyl, (C₁-C₆)alkylcarbonyl, formyl, or (C₁-C₆)alkoxycarbonyl.

In another aspect, the invention provides a method of treating elevated plasma glucose levels in a human comprising administering to the human in need of such treatment a therapeutically effective amount of a compound of Formula (I), or a pharmaceutically-acceptable salt, solvate, or hydrate thereof, wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl; R₁₀ is —H; and R₁₁ is (C₁-C₆)alkylcarbonyl.

In another aspect, the invention provides a method of treating elevated plasma glucose levels in a human comprising administering to the human in need of such treatment a therapeutically effective amount of a compound of Formula (I), or a pharmaceutically-acceptable salt, solvate, or hydrate thereof, wherein M is V₁₀O₂₇OH; X is 5; Y is 5; L₁ is —CH₂—; L₂ is —CH₂—; L₃ is —S(O)₂—; R₁, R₂, R₃, R₄, and R₅ are H; R₆ is phenyl optionally substituted with (C₁-C₆)alkyl.

In another aspect, the invention provides a method of treating ketoacidosis in a human comprising administering to the human in need of such treatment a therapeutically effective amount of a compound of Formula (I), or a pharmaceutically-acceptable salt, solvate, or hydrate thereof, wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and R₇ and R₈ are H.

In another aspect, the invention provides a method of treating ketoacidosis in a human comprising administering to the human in need of such treatment a therapeutically effective amount of a compound of Formula (I), or a pharmaceutically-acceptable salt, solvate, or hydrate thereof, wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; and R₁₀ and R₁₁ are independently H, (C₁-C₆)alkyl, (C₁-C₆)alkylcarbonyl, formyl, or (C₁-C₆)alkoxycarbonyl.

In another aspect, the invention provides a method of treating ketoacidosis in a human comprising administering to the human in need of such treatment a therapeutically effective amount of a compound of Formula (I), or a pharmaceutically-acceptable salt, solvate, or hydrate thereof, wherein M is V₁₀O₂₇OH; X is 5; Y is 5; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl; R₁₀ is —H; and R₁₁ is (C₁-C₆)alkylcarbonyl.

In another aspect, the invention provides a method of treating ketoacidosis in a human comprising administering to the human in need of such treatment a therapeutically effective amount of a compound of Formula (I), or a pharmaceutically-acceptable salt, solvate, or hydrate thereof, wherein M is V₁₀O₂₇OH; X is 5; Y is 5; L₁ is —CH₂—; L₂ is —CH₂—; L₃ is —S(O)₂—; R₁, R₂, R₃, R₄, and R₅ are H; R₆ is phenyl optionally substituted with (C₁-C₆)alkyl.

In another aspect, the invention provides compounds of Formula (I) wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and R₇ and R₈ are H.

In another aspect, the invention provides compounds of Formula (I) wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; and R₆ is (C₁-C₆)alkoxy.

In another aspect, the invention provides compounds of Formula (I) wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; and R₆ is (C₂-C₆)alkenyl.

In another aspect, the invention provides compounds of Formula (I) wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; and R₆ is (C₂-C₆)alkenyloxy.

In another aspect, the invention provides compounds of Formula (I) wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; and R₆ is (C₁-C₆)alkyl.

In another aspect, the invention provides compounds of Formula (I) wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; and R₆ is (C₂-C₆)alkynyl.

In another aspect, the invention provides compounds of Formula (I) wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; and R₆ is (C₂-C₆)alkynyloxy.

In another aspect, the invention provides compounds of Formula (I) wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is phenyl optionally substituted with 1 or 2 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; and R₇ and R₈ are independently H or (C₁-C₆)alkyl.

In another aspect, the invention provides compounds of Formula (I) wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is phenyl optionally substituted with 1 or 2 substituents independently selected from halogen, hydroxy, and NR₇R₈(C₁-C₆)alkyl; and R₇ and R₈ are independently H or (C₁-C₆)alkyl.

In another aspect, the invention provides compounds of Formula (I) wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; and R₆ is phenyl optionally substituted with 1 or 2 substituents independently selected from halogen, hydroxy, and —CH₂NH₂.

In another aspect, the invention provides compounds of Formula (I) wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; and R₆ is phenyl substituted with 1 substituent selected from halogen, hydroxy, and —CH₂NH₂.

In another aspect, the invention provides compounds of Formula (I) wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; and R₆ is (C₁-C₆)haloalkyl.

In another aspect, the invention provides compounds of Formula (I) wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; and R₆ is (C₁-C₆)haloalkoxy.

In another aspect, the invention provides compounds of Formula (I) wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is —NR₇R₈; and R₇ and R₈ are independently H or (C₁-C₆)alkyl.

In another aspect, the invention provides compounds of Formula (I) wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is —CH₂CH₂NR₁₀R₁₁ and R₁₀ and R₁₁ are independently H or (C₁-C₆)alkyl.

In another aspect, the invention provides compounds of Formula (I) wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is —CH₂CH₂NR₁₀R₁₁ and R₁₀ is H; and R₁₁ is (C₁-C₆)alkylcarbonyl.

In another aspect, the invention provides compounds of Formula (I) wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; and R₁₀ and R₁₁ are independently H, (C₁-C₆)alkyl, (C₁-C₆)alkylcarbonyl, formyl, or (C₁-C₆)alkoxycarbonyl.

In another aspect, the invention provides compounds of Formula (I) wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl optionally substituted with 1 or 2 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; R₁₀ is —H; and R₁₁ is (C₁-C₆)alkylcarbonyl.

In another aspect, the invention provides compounds of Formula (I) wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl; R₁₀ is —H; and R₁₁ is (C₁-C₆)alkylcarbonyl.

In another aspect, the invention provides compounds of Formula (I) wherein M is V₁₀O₂₆(OH)₂; X is 4; Y is 4; L₁ is —CH₂—; L₂ is —CH₂—; L₃ is —S(O)₂—; R₁, R₂, R₃, R₄, and R₅ are H; and R₆ is (C₁-C₆)alkoxy.

In another aspect, the invention provides compounds of Formula (I) wherein M is V₁₀O₂₆(OH)₂; X is 4; Y is 4; L₁ is —CH₂—; L₂ is —CH₂—; L₃ is —S(O)₂—; R₁, R₂, R₃, R₄, and R₅ are H; and R₆ is (C₂-C₆)alkenyl.

In another aspect, the invention provides compounds of Formula (I) wherein M is V₁₀O₂₆(OH)₂; X is 4; Y is 4; L₁ is —CH₂—; L₂ is —CH₂—; L₃ is —S(O)₂—; R₁, R₂, R₃, R₄, and R₅ are H; and R₆ is (C₂-C₆)alkenyloxy.

In another aspect, the invention provides compounds of Formula (I) wherein M is V₁₀O₂₆(OH)₂; X is 4; Y is 4; L₁ is —CH₂—; L₂ is —CH₂—; L₃ is —S(O)₂—; R₁, R₂, R₃, R₄, and R₅ are H; and R₆ is (C₁-C₆)alkyl.

In another aspect, the invention provides compounds of Formula (I) wherein M is V₁₀O₂₆(OH)₂; X is 4; Y is 4; L₁ is —CH₂—; L₂ is —CH₂—; L₃ is —S(O)₂—; R₁, R₂, R₃, R₄, and R₅ are H; and R₆ is (C₂-C₆)alkynyl.

In another aspect, the invention provides compounds of Formula (I) wherein M is V₁₀O₂₆(OH)₂; X is 4; Y is 4; L₁ is —CH₂—; L₂ is —CH₂—; L₃ is —S(O)₂—; R₁, R₂, R₃, R₄, and R₅ are H; and R₆ is (C₂-C₆)alkynyloxy.

In another aspect, the invention provides compounds of Formula (I) wherein M is V₁₀O₂₆(OH)₂; X is 4; Y is 4; L₁ is —CH₂—; L₂ is —CH₂—; L₃ is —S(O)₂—; R₁, R₂, R₃, R₄, and R₅ are H; R₆ is phenyl optionally substituted with 1 or 2 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; and R₇ and R₈ are independently H or (C₁-C₆)alkyl.

In another aspect, the invention provides compounds of Formula (I) wherein M is V₁₀O₂₆(OH)₂; X is 4; Y is 4; L₁ is —CH₂—; L₂ is —CH₂—; L₃ is —S(O)₂—; R₁, R₂, R₃, R₄, and R₅ are H; R₆ is phenyl optionally substituted with 1 substituent selected from halogen, hydroxy, and NR₇R₈(C₁-C₆)alkyl; and R₇ and R₈ are independently H or (C₁-C₆)alkyl.

In another aspect, the invention provides compounds of Formula (I) wherein M is V₁₀O₂₆(OH)₂; X is 4; Y is 4; L₁ is —CH₂—; L₂ is —CH₂—; L₃ is —S(O)₂—; R₁, R₂, R₃, R₄, and R₅ are H; and R₆ is phenyl optionally substituted with 1 substituent selected from halogen, hydroxy, and —CH₂NH₂.

In another aspect, the invention provides compounds of Formula (I) wherein M is V₁₀O₂₆(OH)₂; X is 4; Y is 4; L₁ is —CH₂—; L₂ is —CH₂—; L₃ is —S(O)₂—; R₁, R₂, R₃, R₄, and R₅ are H; and R₆ is phenyl substituted with 1 substituent selected from halogen, hydroxy, and —CH₂NH₂.

In another aspect, the invention provides compounds of Formula (I) wherein M is V₁₀O₂₆(OH)₂; X is 4; Y is 4; L₁ is —CH₂—; L₂ is —CH₂—; L₃ is —S(O)₂—; R₁, R₂, R₃, R₄, and R₅ are H; R₆ is phenyl optionally substituted with (C₁-C₆)alkyl.

In another aspect, the invention provides compounds of Formula (I) wherein M is V₁₀O₂₆(OH)₂; X is 4; Y is 4; L₁ is —CH₂—; L₂ is —CH₂—; L₃ is —S(O)₂—; R₁, R₂, R₃, R₄, and R₅ are H; and R₆ is (C₁-C₆)haloalkyl.

In another aspect, the invention provides compounds of Formula (I) wherein M is V₁₀O₂₆(OH)₂; X is 4; Y is 4; L₁ is —CH₂—; L₂ is —CH₂—; L₃ is —S(O)₂—; R₁, R₂, R₃, R₄, and R₅ are H; and R₆ is (C₁-C₆)haloalkoxy.

In another aspect, the invention provides compounds of Formula (I) wherein M is V₁₀O₂₆(OH)₂; X is 4; Y is 4; L₁ is —CH₂—; L₂ is —CH₂—; L₃ is —S(O)₂—; R₁, R₂, R₃, R₄, and R₅ are H; R₆ is NR₇R₈; and R₇ and R₈ are independently H or (C₁-C₆)alkyl.

In another aspect, the invention provides a pharmaceutical composition comprising a compound according to Formula (I), wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and R₇ and R₈ are H, or a pharmaceutically-acceptable solvate, hydrate, or salt thereof, and at least one pharmaceutically-acceptable excipient, diluent or adjuvant thereof.

In another aspect, the invention provides a pharmaceutical composition comprising a compound according to Formula (I), wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; and R₁₀ and R₁₁ are independently H, (C₁-C₆)alkyl, (C₁-C₆)alkylcarbonyl, formyl, or (C₁-C₆)alkoxycarbonyl, or a pharmaceutically-acceptable solvate, hydrate, or salt thereof, and at least one pharmaceutically-acceptable excipient, diluent or adjuvant thereof.

In another aspect, the invention provides a pharmaceutical composition comprising a compound according to Formula (I), wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl; R₁₀ is —H; and R₁₁ is (C₁-C₆)alkylcarbonyl, or a pharmaceutically-acceptable solvate, hydrate, or salt thereof, and at least one pharmaceutically-acceptable excipient, diluent or adjuvant thereof.

In another aspect, the invention provides a pharmaceutical composition comprising a compound according to Formula (I), wherein M is V₁₀O₂₆(OH)₂; X is 4; Y is 4; L₁ is —CH₂—; L₂ is —CH₂—; L₃ is —S(O)₂—; R₁, R₂, R₃, R₄, and R₅ are H; R₆ is phenyl optionally substituted with (C₁-C₆)alkyl, or a pharmaceutically-acceptable solvate, hydrate, or salt thereof, and at least one pharmaceutically-acceptable excipient, diluent or adjuvant thereof.

In another aspect, the invention provides a method of treating type I diabetes in a human comprising administering to the human in need of such treatment a therapeutically effective amount of a compound of Formula (I), or a pharmaceutically-acceptable salt, solvate, or hydrate thereof, wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and R₇ and R₈ are H.

In another aspect, the invention provides a method of treating type I diabetes in a human comprising administering to the human in need of such treatment a therapeutically effective amount of a compound of Formula (I), or a pharmaceutically-acceptable salt, solvate, or hydrate thereof, wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; and R₁₀ and R₁₁ are independently H, (C₁-C₆)alkyl, (C₁-C₆)alkylcarbonyl, formyl, or (C₁-C₆)alkoxycarbonyl.

In another aspect, the invention provides a method of treating type I diabetes in a human comprising administering to the human in need of such treatment a therapeutically effective amount of a compound of Formula (I), or a pharmaceutically-acceptable salt, solvate, or hydrate thereof, wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl; R₁₀ is —H; and R₁₁ is (C₁-C₆)alkylcarbonyl.

In another aspect, the invention provides a method of treating type I diabetes in a human comprising administering to the human in need of such treatment a therapeutically effective amount of a compound of Formula (I), or a pharmaceutically-acceptable salt, solvate, or hydrate thereof, wherein M is V₁₀O₂₆(OH)₂; X is 4; Y is 4; L₁ is —CH₂—; L₂ is —CH₂—; L₃ is —S(O)₂—; R₁, R₂, R₃, R₄, and R₅ are H; R₆ is phenyl optionally substituted with (C₁-C₆)alkyl.

In another aspect, the invention provides a method of treating type II diabetes in a human comprising administering to the human in need of such treatment a therapeutically effective amount of a compound of Formula (I), or a pharmaceutically-acceptable salt, solvate, or hydrate thereof, wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and R₇ and R₈ are H.

In another aspect, the invention provides a method of treating type II diabetes in a human comprising administering to the human in need of such treatment a therapeutically effective amount of a compound of Formula (I), or a pharmaceutically-acceptable salt, solvate, or hydrate thereof, wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; and R₁₀ and R₁₁ are independently H, (C₁-C₆)alkyl, (C₁-C₆)alkylcarbonyl, formyl, or (C₁-C₆)alkoxycarbonyl.

In another aspect, the invention provides a method of treating type II diabetes in a human comprising administering to the human in need of such treatment a therapeutically effective amount of a compound of Formula (I), or a pharmaceutically-acceptable salt, solvate, or hydrate thereof, wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl; R₁₀ is —H; and R₁₁ is (C₁-C₆)alkylcarbonyl.

In another aspect, the invention provides a method of treating type II diabetes in a human comprising administering to the human in need of such treatment a therapeutically effective amount of a compound of Formula (I), or a pharmaceutically-acceptable salt, solvate, or hydrate thereof, wherein M is V₁₀O₂₆(OH)₂; X is 4; Y is 4; L₁ is —CH₂—; L₂ is —CH₂—; L₃ is —S(O)₂—; R₁, R₂, R₃, R₄, and R₅ are H; R₆ is phenyl optionally substituted with (C₁-C₆)alkyl.

In another aspect, the invention provides a method of treating elevated plasma glucose levels in a human comprising administering to the human in need of such treatment a therapeutically effective amount of a compound of Formula (I), or a pharmaceutically-acceptable salt, solvate, or hydrate thereof, wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and R₇ and R₈ are H.

In another aspect, the invention provides a method of treating elevated plasma glucose levels in a human comprising administering to the human in need of such treatment a therapeutically effective amount of a compound of Formula (I), or a pharmaceutically-acceptable salt, solvate, or hydrate thereof, wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; and R₁₀ and R₁₁ are independently H, (C₁-C₆)alkyl, (C₁-C₆)alkylcarbonyl, formyl, or (C₁-C₆)alkoxycarbonyl.

In another aspect, the invention provides a method of treating elevated plasma glucose levels in a human comprising administering to the human in need of such treatment a therapeutically effective amount of a compound of Formula (I), or a pharmaceutically-acceptable salt, solvate, or hydrate thereof, wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl; R₁₀ is —H; and R₁₁ is (C₁-C₆)alkylcarbonyl.

In another aspect, the invention provides a method of treating elevated plasma glucose levels in a human comprising administering to the human in need of such treatment a therapeutically effective amount of a compound of Formula (I), or a pharmaceutically-acceptable salt, solvate, or hydrate thereof, wherein M is V₁₀O₂₆(OH)₂; X is 4; Y is 4; L₁ is —CH₂—; L₂ is —CH₂—; L₃ is —S(O)₂—; R₁, R₂, R₃, R₄, and R₅ are H; R₆ is phenyl optionally substituted with (C₁-C₆)alkyl.

In another aspect, the invention provides a method of treating ketoacidosis in a human comprising administering to the human in need of such treatment a therapeutically effective amount of a compound of Formula (I), or a pharmaceutically-acceptable salt, solvate, or hydrate thereof, wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and R₇ and R₈ are H.

In another aspect, the invention provides a method of treating ketoacidosis in a human comprising administering to the human in need of such treatment a therapeutically effective amount of a compound of Formula (I), or a pharmaceutically-acceptable salt, solvate, or hydrate thereof, wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; and R₁₀ and R₁₁ are independently H, (C₁-C₆)alkyl, (C₁-C₆)alkylcarbonyl, formyl, or (C₁-C₆)alkoxycarbonyl.

In another aspect, the invention provides a method of treating ketoacidosis in a human comprising administering to the human in need of such treatment a therapeutically effective amount of a compound of Formula (I), or a pharmaceutically-acceptable salt, solvate, or hydrate thereof, wherein M is V₁₀O₂₆(OH)₂; X is 4; Y is 4; L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl; R₁₀ is —H; and R₁₁ is (C₁-C₆)alkylcarbonyl.

In another aspect, the invention provides a method of treating ketoacidosis in a human comprising administering to the human in need of such treatment a therapeutically effective amount of a compound of Formula (I), or a pharmaceutically-acceptable salt, solvate, or hydrate thereof, wherein M is V₁₀O₂₆(OH)₂; X is 4; Y is 4; L₁ is —CH₂—; L₂ is —CH₂—; L₃ is —S(O)₂—; R₁, R₂, R₃, R₄, and R₅ are H; R₆ is phenyl optionally substituted with (C₁-C₆)alkyl.

In additional aspects, the invention provides compounds of Formula (II). In particular aspects, the invention provides compounds of Formula (II) wherein L₁ is —CH₂—; L₂ is —CH₂—; L₃ is —C(O)—; R₁, R₂, R₃, R₄, and R₅ are H; R₆ is (C₂-C₆)alkenyloxy, (C₂-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and R₇ and R₈ are H.

In another aspect, the invention provides compounds of Formula (II) wherein L₁ is —CH₂—; L₂ is —CH₂—; L₃ is —C(O)—; R₁, R₂, R₃, R₄, and R₅ are H; and R₆ is (C₁-C₆)alkoxy.

In another aspect, the invention provides compounds of Formula (II) wherein L₁ is —CH₂—; L₂ is —CH₂—; L₃ is —C(O)—; R₁, R₂, R₃, R₄, and R₅ are H; and R₆ is (C₂-C₆)alkenyl.

In another aspect, the invention provides compounds of Formula (II) wherein L₁ is —CH₂—; L₂ is —CH₂—; L₃ is —C(O)—; R₁, R₂, R₃, R₄, and R₅ are H; and R₆ is (C₂-C₆)alkenyloxy.

In another aspect, the invention provides compounds of Formula (II) wherein L₁ is —CH₂—; L₂ is —CH₂—; L₃ is —C(O)—; R₁, R₂, R₃, R₄, and R₅ are H; and R₆ is (C₂-C₆)alkyl.

In another aspect, the invention provides compounds of Formula (II) wherein L₁ is —CH₂—; L₂ is —CH₂—; L₃ is —C(O)—; R₁, R₂, R₃, R₄, and R₅ are H; and R₆ is (C₂-C₆)alkynyl.

In another aspect, the invention provides compounds of Formula (II) wherein L₁ is —CH₂—; L₂ is —CH₂—; L₃ is —C(O)—; R₁, R₂, R₃, R₄, and R₅ are H; and R₆ is (C₂-C₆)alkynyloxy.

In another aspect, the invention provides compounds of Formula (II) wherein L₁ is —CH₂—; L₂ is —CH₂—; L₃ is —C(O)—; R₁, R₂, R₃, R₄, and R₅ are H; R₆ is phenyl optionally substituted with 1 or 2 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, —NR₇R₈, and NR₇R₈(C₁-C₆)alkyl; and R₇ and R₈ are independently H or (C₁-C₆)alkyl.

In another aspect, the invention provides compounds of Formula (II) wherein L₁ is —CH₂—; L₂ is —CH₂—; L₃ is —C(O)—; R₁, R₂, R₃, R₄, and R₅ are H; R₆ is phenyl optionally substituted with 1 or 2 substituents independently selected from halogen, hydroxy, and NR₇R₈(C₁-C₆)alkyl; and R₇ and R₈ are independently H or (C₁-C₆)alkyl.

In another aspect, the invention provides compounds of Formula (II) wherein L₁ is —CH₂—; L₂ is —CH₂—; L₃ is —C(O)—; R₁, R₂, R₃, R₄, and R₅ are H; and R₆ is phenyl optionally substituted with 1 or 2 substituents independently selected from halogen, hydroxy, and —CH₂NH₂.

In another aspect, the invention provides compounds of Formula (II) wherein L₁ is —CH₂—; L₂ is —CH₂—; L₃ is —C(O)—; R₁, R₂, R₃, R₄, and R₅ are H; and R₆ is phenyl substituted with 1 substituent selected from halogen, hydroxy, and —CH₂NH₂.

In another aspect, the invention provides compounds of Formula (II) wherein L₁ is —CH₂—; L₂ is —CH₂—; L₃ is —C(O)—; R₁, R₂, R₃, R₄, and R₅ are H; and R₆ is (C₁-C₆)haloalkyl.

In another aspect, the invention provides compounds of Formula (II) wherein L₁ is —CH₂—; L₂ is —CH₂—; L₃ is —C(O)—; R₁, R₂, R₃, R₄, and R₅ are H; and R₆ is (C₁-C₆)haloalkoxy.

In another aspect, the invention provides compounds of Formula (II) wherein L₁ is —CH₂—; L₂ is —CH₂—; L₃ is —C(O)—; R₁, R₂, R₃, R₄, and R₅ are H; R₆ is —NR₇R₈; and R₇ and R₈ are independently H or (C₁-C₆)alkyl.

In another aspect, the invention provides compounds of Formula (II) wherein L₁ is —CH₂—; L₂ is —CH₂—; L₃ is —C(O)—; R₁, R₂, R₃, R₄, and R₅ are H; R₆ is —CH₂CH₂NR₁₀R₁₁ and R₁₀ and R₁₁ are independently H or (C₁-C₆)alkyl.

In another aspect, the invention provides compounds of Formula (II) wherein L₁ is —CH₂—; L₂ is —CH₂—; L₃ is —C(O)—; R₁, R₂, R₃, R₄, and R₅ are H; R₆ is —CH₂CH₂NR₁₀R₁₁ and R₁₀ is H; and R₁₁ is (C₁-C₆)alkylcarbonyl.

In another aspect, the invention provides compounds of Formula (II) wherein L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; R₇ and R₈ are independently H or (C₁-C₆)alkyl; and R₁₀ and R₁₁ are independently H, (C₁-C₆)alkyl, (C₁-C₆)alkylcarbonyl, formyl, or (C₁-C₆)alkoxycarbonyl.

In another aspect, the invention provides compounds of Formula (II) wherein L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl optionally substituted with 1 or 2 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, —NR₇R₈, and NR₇R₈(C₁-C₆)alkyl; R₇ and R₈ are independently H or (C₁-C₆)alkyl; R₁₀ is —H; and R₁₁ is (C₁-C₆)alkylcarbonyl.

In another aspect, the invention provides compounds of Formula (II) wherein L₁ is —CH₂—; L₂ is —CH₂—; L₃ is —C(O)—; R₁, R₂, R₃, R₄, and R₅ are H; R₆ is —CH(R₉)NR₁₀R₁₁; R₉ is phenyl; R₁₀ is —H; and R₁₁ is (C₁-C₆)alkylcarbonyl.

In another aspect, the invention provides compounds of Formula (II) wherein L₁ is —CH₂—; L₂ is —CH₂—; L₃ is —S(O)₂—; R₁, R₂, R₃, R₄, and R₅ are H; and R₆ is (C₁-C₆)alkoxy.

In another aspect, the invention provides compounds of Formula (II) wherein L₁ is —CH₂—; L₂ is —CH₂—; L₃ is —S(O)₂—; R₁, R₂, R₃, R₄, and R₅ are H; and R₆ is (C₂-C₆)alkenyl.

In another aspect, the invention provides compounds of Formula (II) wherein L₁ is —CH₂—; L₂ is —CH₂—; L₃ is —S(O)₂—; R₁, R₂, R₃, R₄, and R₅ are H; and R₆ is (C₂-C₆)alkenyloxy.

In another aspect, the invention provides compounds of Formula (II) wherein L₁ is —CH₂—; L₂ is —CH₂—; L₃ is —S(O)₂—; R₁, R₂, R₃, R₄, and R₅ are H; and R₆ is (C₂-C₆)alkyl.

In another aspect, the invention provides compounds of Formula (II) wherein L₁ is —CH₂—; L₂ is —CH₂—; L₃ is —S(O)₂—; R₁, R₂, R₃, R₄, and R₅ are H; and R₆ is (C₂-C₆)alkynyl.

In another aspect, the invention provides compounds of Formula (II) wherein L₁ is —CH₂—; L₂ is —CH₂—; L₃ is —S(O)₂—; R₁, R₂, R₃, R₄, and R₅ are H; and R₆ is (C₂-C₆)alkynyloxy.

In another aspect, the invention provides compounds of Formula (II) wherein L₁ is —CH₂—; L₂ is —CH₂—; L₃ is —S(O)₂—; R₁, R₂, R₃, R₄, and R₅ are H; R₆ is phenyl optionally substituted with 1 or 2 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, —NR₇R₈, and NR₇R₈(C₁-C₆)alkyl; and R₇ and R₈ are independently H or (C₁-C₆)alkyl.

In another aspect, the invention provides compounds of Formula (II) wherein L₁ is —CH₂—; L₂ is —CH₂—; L₃ is —S(O)₂—; R₁, R₂, R₃, R₄, and R₅ are H; R₆ is phenyl optionally substituted with 1 substituent selected from halogen, hydroxy, and NR₇R₈(C₁-C₆)alkyl; and R₇ and R₈ are independently H or (C₁-C₆)alkyl.

In another aspect, the invention provides compounds of Formula (II) wherein L₁ is —CH₂—; L₂ is —CH₂—; L₃ is —S(O)₂—; R₁, R₂, R₃, R₄, and R₅ are H; and R₆ is phenyl optionally substituted with 1 substituent selected from halogen, hydroxy, and —CH₂NH₂.

In another aspect, the invention provides compounds of Formula (II) wherein L₁ is —CH₂—; L₂ is —CH₂—; L₃ is —S(O)₂—; R₁, R₂, R₃, R₄, and R₅ are H; and R₆ is phenyl substituted with 1 substituent selected from halogen, hydroxy, and —CH₂NH₂.

In another aspect, the invention provides compounds of Formula (II) wherein L₁ is —CH₂—; L₂ is —CH₂—; L₃ is —S(O)₂—; R₁, R₂, R₃, R₄, and R₅ are H; R₆ is phenyl optionally substituted with (C₁-C₆)alkyl.

In another aspect, the invention provides compounds of Formula (II) wherein L₁ is —CH₂—; L₂ is —CH₂—; L₃ is —S(O)₂—; R₁, R₂, R₃, R₄, and R₅ are H; and R₆ is (C₁-C₆)haloalkyl.

In another aspect, the invention provides compounds of Formula (II) wherein L₁ is —CH₂—; L₂ is —CH₂—; L₃ is —S(O)₂—; R₁, R₂, R₃, R₄, and R₅ are H; and R₆ is (C₁-C₆)haloalkoxy.

In another aspect, the invention provides compounds of Formula (II) wherein L₁ is —CH₂—; L₂ is —CH₂—; L₃ is —S(O)₂—; R₁, R₂, R₃, R₄, and R₅ are H; R₆ is —NR₇R₈; and R₇ and R₈ are independently H or (C₁-C₆)alkyl.

### DEFINITIONS

As used throughout this specification and the appended claims, the following terms have the following meanings:

The term “(C₂-C₆)alkenyl” as used herein, means a straight or branched chain hydrocarbon containing from 2 to 6 carbons and containing at least one carbon-carbon double bond. Representative examples of (C₂-C₆)alkenyl include, but are not limited to, ethenyl, 2-propenyl(allyl), 2-methyl-2-propenyl, 3-butenyl, 4-pentenyl, 5-hexenyl.

The term “(C₂-C₆)alkenyloxy” as used herein, means a (C₂-C₆)alkenyl group, as defined herein, appended to the parent molecular moiety through an oxygen atom.

The term “(C₁-C₆)alkoxy” as used herein, means an alkyl group, as defined herein, appended to the parent molecular moiety through an oxygen atom. Representative examples of (C₁-C₆)alkoxy include, but are not limited to, methoxy, ethoxy, propoxy, isopropoxy, n-butoxy, sec-butoxy, tert-butoxy, pentyloxy, 2-pentyloxy, isopentyloxy, neopentyloxy, hexyloxy, 2-hexyloxy, 3-hexyloxy, and 3-methylpentyloxy.

The term “(C₁-C₆)alkoxy(C₁-C₆)alkyl” as used herein, means a (C₁-C₆)alkoxy group, as defined herein, appended to the parent molecular moiety through a (C₁-C₆)alkyl group, as defined herein.

The term “(C₁-C₆)alkoxycarbonyl” as used herein, means a (C₁-C₆)alkoxy group, as defined herein, appended to the parent molecular moiety through a carbonyl group, as defined herein. Representative examples of (C₁-C₆)alkoxycarbonyl include, but are not limited to, methoxycarbonyl, ethoxycarbonyl, and tert-butoxycarbonyl.

The term “(C₁-C₆)alkyl” as used herein, means a straight or branched chain hydrocarbon containing from 1 to 6 carbon atoms. Representative examples of (C₁-C₆)alkyl include, but are not limited to, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl.

The term “(C₁-C₆)alkylcarbonyl” as used herein, means a (C₁-C₆)alkyl group, as defined herein, appended to the parent molecular moiety through a carbonyl group, as defined herein. Representative examples of (C₁-C₆)alkylcarbonyl include, but are not limited to, acetyl, 1-oxopropyl, 2,2-dimethyl-1-oxopropyl, 1-oxobutyl, and 1-oxopentyl.

The term “(C₁-C₆)alkylcarbonyloxy” as used herein, means a (C₁-C₆)alkylcarbonyl group, as defined herein, appended to the parent molecular moiety through an oxygen atom. Representative examples of (C₁-C₆)alkylcarbonyloxy include, but are not limited to, acetyloxy, ethylcarbonyloxy, and tert-butylcarbonyloxy.

The term “(C₁-C₆)alkylene” means a divalent group derived from a straight or branched chain hydrocarbon of from 1 to 6 carbon atoms. Representative examples of (C₁-C₆)alkylene include, but are not limited to, —CH₂—, —CH(CH₃)—, —C(CH₃)₂—, —CH₂CH₂—, —CH₂CH₂CH₂—, —CH₂CH₂CH₂CH₂—, and —CH₂CH(CH₃)CH₂—.

The term “(C₁-C₆)alkylthio” as used herein, means a (C₁-C₆)alkyl group, as defined herein, appended to the parent molecular moiety through a sulfur atom. Representative examples of (C₁-C₆)alkylthio include, but are not limited, methylthio, ethylthio, tert-butylthio, and hexylthio.

The term “(C₁-C₆)alkylthio(C₁-C₆)alkyl” as used herein, means a (C₁-C₆)alkylthio group, as defined herein, appended to the parent molecular moiety through a (C₁-C₆)alkyl group, as defined herein. Representative examples of (C₁-C₆)alkylthio include, but are not limited, methylthio, ethylthio, tert-butylthio, and hexylthio.

The term “(C₂-C₆)alkynyl” as used herein, means a straight or branched chain hydrocarbon group containing from 2 to 6 carbon atoms and containing at least one carbon-carbon triple bond. Representative examples of (C₂-C₆)alkynyl include, but are not limited, to acetylenyl, 1-propynyl, 2-propynyl, 3-butynyl, 2-pentynyl, and 1-butynyl.

The term “(C₂-C₆)alkynyloxy” as used herein, means a (C₂-C₆)alkynyl group, as defined herein, appended to the parent molecular moiety through an oxygen atom.

The term “aryl” as used herein, means a phenyl or naphthyl group.

The term “carbonyl” as used herein, means a —C(O)— group.

The term “carboxy” as used herein, means a —CO₂H group.

The term “carboxy(C₁-C₆)alkyl” as used herein, means a carboxy group, as defined herein, is attached to the parent molecular moiety through a (C₁-C₆)alkyl group.

The term “(C₁-C₆)alkoxycarbonyl” as used herein, means a (C₁-C₆)alkoxy group, as defined herein, appended to the parent molecular moiety through a carbonyl group, as defined herein.

The term “(C₁-C₆)alkoxycarbonyl(C₁-C₆)alkyl” as used herein, means a (C₁-C₆)alkoxycarbonyl group, as defined herein, appended to the parent molecular moiety through a (C₁-C₆)alkyl group, as defined herein.

The term “cyano” as used herein, means a —CN group.

The term “(C₃-C₇)cycloalkyl” as used herein, means a saturated cyclic hydrocarbon group containing from 3 to 7 carbons, examples of (C₃-C₇)cycloalkyl include cyclopropyl, cyclobutyl, cyclopentyl, cyclohexyl, and cycloheptyl

The term “(C₃-C₇)cycloalkyl(C₁-C₆)alkyl” as used herein, means a (C₃-C₇)cycloalkyl group, as defined herein, appended to the parent molecular moiety through a (C₁-C₆)alkyl group, as defined herein. Representative examples of (C₃-C₇)cycloalkyl(C₁-C₆)alkyl include, but are not limited to, cyclopropylmethyl, 2-cyclobutylethyl, cyclopentylmethyl, cyclohexylmethyl, and 2-cyclohexylethyl.

The term “ethylenedioxy” as used herein, means a —O(CH₂)₂O— group wherein the oxygen atoms of the ethylenedioxy group are attached to the parent molecular moiety through two adjacent carbon atoms, for example Rₐ and R_{b} or R_{b} and R_{c} or R_{c}, and R_{d} or R_{d} and Rₑ.

The term “formyl” as used herein, means a —C(O)H group.

The term “halo” or “halogen” as used herein, means —Cl, —Br, —I or —F.

The term “halo(C₁-C₄)alkoxy” as used herein, means at least one halogen, as defined herein, appended to the parent molecular moiety through a (C₁-C₄)alkoxy group, as defined herein. Representative examples of halo(C₁-C₄)alkoxy include, but are not limited to, chloromethoxy, 2-fluoroethoxy, trifluoromethoxy, and pentafluoroethoxy.

The term “halo(C₁-C₄)alkyl” as used herein, means at least one halogen, as defined herein, appended to the parent molecular moiety through a (C₁-C₄)alkyl group, as defined herein. Representative examples of halo(C₁-C₈)alkyl include, but are not limited to, chloromethyl, 2-bromoethyl, 2-chloroethyl, 2-fluoroethyl, 2-iodoethyl, trichloromethyl, trifluoromethyl, pentafluoroethyl.

The term “heteroaryl,” as used herein, means a monocyclic heteroaryl or a bicyclic heteroaryl. The monocyclic heteroaryl is a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, or a monocyclic heteroaryl fused to a monocyclic heteroaryl. The bicyclic heteroaryl is connected to the parent molecular moiety through any carbon atom or any nitrogen atom contained within the bicyclic heteroaryl. Representative examples of bicyclic heteroaryl include, but are not limited to, benzimidazolyl, benzofuranyl, benzothienyl, benzoxadiazolyl, cinnolinyl, dihydroquinolinyl, dihydroisoquinolinyl, furopyridinyl, indazolyl, indolyl, isoquinolinyl, naphthyridinyl, quinolinyl, tetrahydroquinolinyl, and thienopyridinyl.

The term “hydroxy” as used herein, means an —OH group.

The term “hydroxy(C₁-C₆)alkyl” as used herein, means at least one hydroxy group, as defined herein, is appended to the parent molecular moiety through a (C₁-C₆)alkyl group, as defined herein. Representative examples of hydroxy(C₁-C₆)alkyl include, but are not limited to, hydroxymethyl, 2-hydroxyethyl, 3-hydroxypropyl, and 2,3-dihydroxypentyl.

The term “mercapto” as used herein, means a —SH group.

The term “methylenedioxy” as used herein, means a —OCH₂O— group wherein the oxygen atoms of the methylenedioxy are attached to the parent molecular moiety through two adjacent carbon atoms, for example, Rₐ and R_{b} or R_{b} and R_{c} or R_{c}, and R_{d} or R_{d} and Rₑ.

The term “nitro” as used herein, means a —NO₂ group.

The term “NR₇R₈” as used herein, means two groups, R₇ and R₈, which are appended to the parent molecular moiety through a nitrogen atom. R₇ and R₈ are each independently H or (C₁-C₆)alkyl. Representative examples of NR₇R₈ include, but are not limited to, amino, methylamino, dimethylamino, ethylamino, and diethylamino.

The term “NR₇R₈ (C₁-C₆)alkyl” as used herein, means a NR₇R₈ group, as defined herein, appended to the parent molecular moiety through a (C₁-C₆)alkyl group, as defined herein.

The term “NR₇R₈carbonyl” used herein, means a NR₇R₈ group, as defined herein, appended to the parent molecular moiety through a carbonyl group, as defined herein.

The term “NR₇R₈carbonyl(C₁-C₆)alkyl” used herein, means a NR₇R₈carbonyl group, as defined herein, appended to the parent molecular moiety through a (C₁-C₆)alkyl group, as defined herein.

The term “NH₂C(═NH)NH(C₁-C₆)alkyl” as used herein, means a NH₂C(═NH)NH— group appended to the parent molecular moiety through a (C₁-C₆)alkyl group, as defined herein.

The term “thio(C₁-C₆)alkyl” as used herein, means a sulfur atom appended to the parent molecular moiety through a (C₁-C₆)alkyl group, as defined herein. Representative examples of thio(C₁-C₆)alkyl include, but are not limited, thiomethyl, 2-thioethyl, 3-thiopropyl, 2-thiopropyl, and 4-thiobutyl.

Compounds of the present invention were named by either ACD/ChemSketch version 8.0 (developed by Advanced Chemistry Development, Inc., Toronto, ON, Canada) or by Chemdraw Ultra version 10.0.

Compounds of the invention can exist as stereoisomers, wherein asymmetric or chiral centers are present. Stereoisomers are designated (R) or (S), depending on the configuration of substituents around the chiral carbon atom. The terms (R) and (S) used herein are configurations as defined in IUPAC 1974 Recommendations for Section E, Fundamental Stereochemistry, Pure Appl. Chem., (1976), 45: 13-30. The present invention contemplates various stereoisomers and mixtures thereof and are specifically included within the scope of this invention. Stereoisomers include enantiomers, diastereomers, and mixtures of enantiomers or diastereomers. In particular, the stereochemistry at R₉ may independently be either (R) or (S). Individual stereoisomers of compounds of the present invention may be prepared synthetically from commercially available starting materials which contain asymmetric or chiral centers or by preparation of racemic mixtures followed by resolution, a technique well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) direct separation of the mixture of optical enantiomers on chiral chromatographic columns, or (3) formation of a diastereomeric salt followed by selective recrystallization of one of the diastereomeric salts.

Salts of the invention comprise one or more ammonium cations and a negatively charged vanadium metal complex or vanadate. If the overall charge of the ammonium-vanadate salt is positively charged, the salt my further comprise a counter ion, e.g. F⁻, Cl⁻, Br⁻, I⁻, OH—, or any pharmaceutically acceptable organic or inorganic ionic species which carries a negative charge. If the overall charge of the ammonium-vanadate salt is negatively charged, the salt may further comprise a counter ion which is positively charged. Positively charged counter ions typically comprise metals from alkali- or earth alkali metals, such as sodium, potassium, magnesium, calcium, as well as other positively charged ions such as ammonium or any pharmaceutically acceptable organic or inorganic ionic species which carries a positive charge. In aqueous solutions, oxovanadates [VO₄]³⁻, [HVO₄]²⁻, [H₂VO₄]³⁻, [V₂O₇]⁴⁻, [HV₂O₇]³⁻, [V₃O₉]³⁻, [V₄O₁₂]⁴⁻, [V₁₀O₂₈]⁶⁻, [V₁₀O₂₇OH]⁵⁻, and [V₁₀O₂₆(OH)₂]⁴⁻ are present in reliably detectable proportions depending on the pH of the solution.

Hydrates of compounds of Formula (I), and their uses, are within the scope of this invention.

Compounds of the invention are useful as pharmaceutical agents, and can be provided as pharmaceutical compositions. The pharmaceutical compositions can be manufactured in a manner that is itself known, e.g., by means of a conventional mixing, dissolving, granulating, dragee-making, levigating, emulsifying, encapsulating, entrapping or lyophilizing processes.

Pharmaceutical compositions can be formulated in conventional manner using one or more physiologically acceptable carriers comprising excipients and auxiliaries that facilitate processing of the active compounds into preparations that can be used pharmaceutically. Proper formulation is dependent upon the route of administration chosen.

For injection, the compounds of the invention can be formulated in appropriate aqueous solutions, such as physiologically compatible buffers such as Hanks's solution, Ringer's solution, or physiological saline buffer. For transmucosal and transcutaneous administration, penetrants appropriate to the barrier to be permeated are used in the formulation. Such penetrants are generally known in the art.

For oral administration, the compounds of the invention can be formulated readily by combining the active compounds with pharmaceutically acceptable carriers well-known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration. For buccal administration, the compositions can take the form of tablets or lozenges formulated in conventional manner.

For administration by inhalation, the compounds of the invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compounds of the invention can be formulated for parenteral administration by injection, e.g., by bolus injection or continuous infusion.

Formulations for injection can be presented in unit dosage form, e.g. in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions.

Suitable lipophilic solvents or vehicles include fatty oils such as sesame oil, or synthetic fatty acid esters, such as ethyloleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the compounds to allow for the preparation of highly concentrated solutions. Alternatively, the active ingredient can be in powder form for constitution with a suitable vehicle, e.g., sterile pyrogen-free water, before use. The compounds of the invention can also be formulated in rectal compositions such as suppositories or retention enemas, e.g. containing conventional suppository bases such as cocoa butter or other glycerides.

In addition to the formulations described previously, the compounds of the invention can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in acceptable oil) or ion exchange resins, or as sparingly soluble derivatives, for example, as a sparingly soluble salt.

A pharmaceutical carrier for hydrophobic compounds of the invention is a cosolvent system comprising benzyl alcohol, a nonpolar surfactant, a water-miscible organic polymer, and an aqueous phase. The cosolvent system can be the VPD co-solvent system. VPD is a solution of 3% w/v benzyl alcohol, 8% w/v of the nonpolar surfactant polysorbate 80, and 65% w/v polyethylene glycoL300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system can be varied considerably without destroying its solubility and toxicity characteristics. Furthermore, the identity of the co-solvent components can be varied: for example, other low-toxicity nonpolar surfactants can be used instead of polysorbate 80; the fraction size of polyethylene glycol can be varied; other biocompatible polymers can replace polyethylene glycol, e.g. polyvinyl pyrrolidone; and other sugars or polysaccharides can substitute for dextrose.

Alternatively, other delivery systems for hydrophobic pharmaceutical compounds can be employed. Liposomes and emulsions are well known examples of delivery vehicles or carriers for hydrophobic drugs. Certain organic solvents such as dimethylsulfoxide also can be employed, although usually at the cost of greater toxicity. Additionally, the compounds of the invention can be delivered using a sustained-release system, such as semipermeable matrices of solid hydrophobic polymers containing the therapeutic agent. Various sustained-release materials have been established and are well known by those skilled in the art. Sustained-release capsules can, depending on their chemical nature, release the compounds for a few weeks up to over 100 days. Depending on the chemical nature and the biological stability of the therapeutic reagent, additional strategies for protein and nucleic acid stabilization can be employed.

The pharmaceutical compositions of the invention also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The compounds of the invention can be provided as salts with pharmaceutically compatible counterions. Pharmaceutically compatible salts can be formed with many acids, including but not limited to hydrochloric, sulfuric, acetic, lactic, tartaric, malic, succinic, phosphoric, hydrobromic, sulfinic, formic, toluenesulfonic, methanesulfonic, benzenesulfonic, nitric, benzoic, citric, tartaric, maleic, hydroiodic, alkanoic such as acetic, and HOOC—(CH₂)ₙ—CH₃ where n is 1-4, and the like. Salts tend to be more soluble in aqueous or other protonic solvents that are the corresponding free base forms. Non-toxic pharmaceutical base addition salts include salts of bases such as sodium, potassium, calcium, ammonium, and the like. Those skilled in the art will recognize a wide variety of non-toxic pharmaceutically acceptable addition salts.

Pharmaceutical compositions of the compounds of the invention can be formulated and administered through a variety of means, including systemic, localized, or topical administration. Techniques for formulation and administration can be found in “Remington's Pharmaceutical Sciences,” Mack Publishing Co., Easton, Pa. The mode of administration can be selected to maximize delivery to a desired target site in the body. Suitable routes of administration can, for example, include oral, rectal, transmucosal, transcutaneous, or intestinal administration; potential delivery, including intramuscular, subcutaneous, intramedullary injections, as well as intrathecal, direct intraventricular, intravenous, intraperitoneal, intranasal, or intraocular injections.

Alternatively, one can administer the compound in a local rather than systemic manner, for example, via injection of the compound directly into a specific tissue, often in a depot or sustained release formulation.

Pharmaceutical compositions suitable for use include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For administration to non-human animals, the drug or a pharmaceutical composition containing the drug may also be added to the animal feed or drinking water. It will be convenient to formulate animal feed and drinking water products with a predetermined dose of the drug so that the animal takes in an appropriate quantity of the drug along with its diet. It will also be convenient to add a premix containing the drug to the feed or drinking water approximately immediately prior to consumption by the animal.

Compounds of the invention have certain pharmacological properties. Such properties include, but are not limited to oral bioavailability, low toxicity, low serum protein binding and desirable in vitro and in vivo half-lives. Assays may be used to predict these desirable pharmacological properties. Assays used to predict bioavailability include transport across human intestinal cell monolayers, including Caco-2 cell monolayers. Serum protein binding may be predicted from albumin binding assays. Such assays are described in a review by Oravcová et al. (1996, Journal of Chromatography B-Biomedical Applications 677:1-28). Compound half-life is inversely proportional to the frequency of dosage of a compound. In vitro half-lives of compounds of the invention may be predicted from assays of microsomal half-life as described by Kuhnz and Gieschen (1998, Drug Metabolism and Disposition 26:1120-1127).

Toxicity and therapeutic efficacy of such compounds can be determined by conventional pharmaceutical procedures in cell cultures or experimental animals, e.g. for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds that exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of the patient's condition. (See, e.g. Fing et al, 1975, in THE PHARMACOLOGICAL BASIS OF THERAPEUTICS, Ch. 1, p. 1).

Dosage amount and interval can be adjusted individually to provide plasma levels of the active moiety that are sufficient to maintain bacterial cell growth-inhibitory effects. Usual patient dosages for systemic administration range from 100-2000 mg/day. Stated in terms of patient body surface areas, usual dosages range from 50-910 mg/m²/day. Usual average plasma levels should be maintained within 0.1-1000 μM. In cases of local administration or selective uptake, the effective local concentration of the compound cannot be related to plasma concentration.

As used herein, the term “therapeutically effective amount” means the amount of a compound that, when administered to a mammal, in particular a human, for treating a disease, is sufficient to effect such treatment for the disease. The “therapeutically effective amount” will vary depending on the compound, the disease and its severity and the age, weight, or other relevant characteristics of the mammal to be treated.

The compounds of the invention may be prepared by use of known chemical reactions and procedures. Representative methods for synthesizing compounds of the invention are presented below. It is understood that the nature of the substituents required for the desired target compound often determines the preferred method of synthesis. All variable groups of these methods are as described in the generic description if they are not specifically defined below.

Solid-phase manipulations were performed in polypropylene syringes fitted with a polyethylene porous disc. Solvents and soluble reagents were removed by filtration.

Representative compounds of the invention include pharmaceutically acceptable acid and base addition salts. In addition, if a compound is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds.

Compounds of Formula (I) are useful as pharmaceutical agents, and can be provided as pharmaceutical compositions. The pharmaceutical compositions can be manufactured in a manner that is itself known, e.g., by means of a conventional mixing, dissolving, granulating, dragee-making, levigating, emulsifying, encapsulating, entrapping or lyophilizing processes.

Pharmaceutical compositions can be formulated in conventional manner using one or more physiologically acceptable carriers comprising excipients and auxiliaries that facilitate processing of the active compounds into preparations that can be used pharmaceutically. Proper formulation is dependent upon the route of administration chosen.

For injection, the compounds prepared according to the methods of the invention can be formulated in appropriate aqueous solutions, such as physiologically compatible buffers such as Hanks's solution, Ringer's solution, or physiological saline buffer. For transmucosal and transcutaneous administration, penetrants appropriate to the barrier to be permeated are used in the formulation. Such penetrants are generally known in the art.

For oral administration, the compounds can be formulated readily by combining the active compounds with pharmaceutically acceptable carriers well-known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained with solid excipient, optionally grinding a resulting mixture, and processing the mixture of granules, after adding suitable auxiliaries, if desired, to obtain tablets or dragee cores. Suitable excipients are, in particular, fillers such as cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration. For buccal administration, the compositions can take the form of tablets or lozenges formulated in conventional manner.

For administration by inhalation, the compounds prepared according to the methods of the invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The compounds can be formulated for parenteral administration by injection, e.g. by bolus injection or continuous infusion. Formulations for injection can be presented in unit dosage form, e.g. in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyloleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the compounds to allow for the preparation of highly concentrated solutions. Alternatively, the active ingredient can be in powder form for constitution with a suitable vehicle, e.g., sterile pyrogen-free water, before use. The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in acceptable oil) or ion exchange resins, or as sparingly soluble derivatives, for example, as a sparingly soluble salt.

Hydrophobic materials include a cosolvent system comprising benzyl alcohol, a nonpolar surfactant, a water-miscible organic polymer, and an aqueous phase. The cosolvent system can be the VPD co-solvent system. VPD is a solution of 3% w/v benzyl alcohol, 8% w/v of the nonpolar surfactant polysorbate 80, and 65% w/v polyethylene glycoL300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system can be varied considerably without destroying its solubility and toxicity characteristics. Furthermore, the identity of the co-solvent components can be varied: for example, other low-toxicity nonpolar surfactants can be used instead of polysorbate 80; the fraction size of polyethylene glycol can be varied; other biocompatible polymers can replace polyethylene glycol, e.g. polyvinyl pyrrolidone; and other sugars or polysaccharides can substitute for dextrose.

Alternatively, other delivery systems for hydrophobic pharmaceutical compounds can be employed. Liposomes and emulsions are well known examples of delivery vehicles or carriers for hydrophobic drugs. Certain organic solvents such as dimethylsulfoxide also can be employed, although usually at the cost of greater toxicity. Additionally, the compounds can be delivered using a sustained-release system, such as semipermeable matrices of solid hydrophobic polymers containing the therapeutic agent. Various sustained-release materials have been established and are well known by those skilled in the art. Sustained-release capsules can, depending on their chemical nature, release the compounds for a few weeks up to over 100 days. Depending on the chemical nature and the biological stability of the therapeutic reagent, additional strategies for protein and nucleic acid stabilization can be employed.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Pharmaceutical compositions prepared according to the methods of the invention can be formulated and administered through a variety of means, including systemic, localized, or topical administration. Techniques for formulation and administration can be found in “Remington's Pharmaceutical Sciences,” Mack Publishing Co., Easton, Pa. The mode of administration can be selected to maximize delivery to a desired target site in the body. Suitable routes of administration can, for example, include oral, rectal, transmucosal, transcutaneous, or intestinal administration; potential delivery, including intramuscular, subcutaneous, intramedullary injections, as well as intrathecal, direct intraventricular, intravenous, intraperitoneal, intranasal, or intraocular injections.

Pharmaceutical compositions suitable for use include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For administration to non-human animals, the drug or a pharmaceutical composition containing the drug may also be added to the animal feed or drinking water. It will be convenient to formulate animal feed and drinking water products with a predetermined dose of the drug so that the animal takes in an appropriate quantity of the drug along with its diet. It will also be convenient to add a premix containing the drug to the feed or drinking water approximately immediately prior to consumption by the animal.

Toxicity and therapeutic efficacy of the pharmaceutical composition comprising compounds of Formula (I) may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which may be expressed as the ratio LD₅₀/ED₅₀.

The disclosures in this application of all articles and references, including patents, are incorporated herein by reference.

Preparation of Mouse Adipose Tissue Membranes.

Internal adipose tissue was obtained from Swiss mice weighing between 20 to grams. The tissue was cut and homogenized in HES buffer (25 mmol/l HEPES, 2 mmol/l EDTA, 255 mmol/l sucrose) with antiproteases (1 μmol/l pepstatin, 1 μmol/l leupeptin, 0.14 trypsin inhibitor units per ml aproptinin and 1 mmol/PMSF). The lysates were then centrifuged at 5000 g at 4° C. for 15 min to eliminate the fat cake and non-homogenized material, and supernatants were collected and centrifuged at 200000 g for 2 h at 4° C. Pelleted membranes were resuspended in 30 mmol/l HEPES and stored at −80° C. until use. Protein concentrations were measured by the Bradford method with γ-globulin as standard.

Fluorimetric Detection of SSAO-Mediated H₂O₂ Formation.

The SSAO activity of human recombinant VAP-1 (0.1 μg prot/assay) and mouse adipose tissue membranes (1 μg prot/assay) was measured using Amplex Red Reagent, a highly sensitive and stable probe for H₂O₂. The reaction was performed in 200 μL of 0.2 mol/l phosphate buffer at pH 7.4 for 50 min at 37° C. in black non-phosphorescent microplates (Nunc). Catalytic reaction was initiated by the addition of the amines indicated as putative SSAO substrates and H₂O₂-detecting mixture containing horseradish peroxidase and Amplex Red, as previously described. Fluorescence intensity was measured (excitation, 545 nm, 590 nm, Bio-Tek fluorescence plate reader) and H₂O₂ concentration was calculated form calibration curves generated by serial dilutions of standard H₂O₂. Fluorescence readings were performed every 5 min. Blank values were measured in assays pre-incubated with 250 μM semicarbazide for 20 min to totally inhibit SSAO activity, and these values were subtracted from the total amount of H₂O₂ formed. To test amines as putative SSAO inhibitors, they were pre-incubated for 20 min with semicarbazide, and their percentage of inhibition was calculated by referring to SSAO activity produced by benzylamine. The kinetic parameters Kₘ and Vₘₐₓ were calculated using appropriate non-linear curve-fitting formula based on the Michaelis-Menten equation and using GraphPad Prism 4.0 software.

The following abbreviations are used herein: ACN for acetonitrile; Alloc for allyloxycarbonyl; Boc for t-butyloxycarbonyl; Bz for benzyl; TFA for trifluoroacetic acid; THF for tetrahydrofuran; MeOH for methanol; F-moc for 9-fluorenylmethyloxycarbonyl; DMF for dimethylformamide; DCM for methylenechloride; DIEA for N,N-diisopropylethylamine; CDI for 1,1′-carbonyldiimidazole; HOBt for 1-hydroxybenzotriazole; HOAt for 1-Hydroxy-7-azabentriazole; DIPCDI for N,N′-diisopropylcarbodiimide; HATU for (N-dimethylamino)-1H-1,2,3-triazolo(4,5-b)pyridine-1-ylmethylene)-N-ethylmethanominium hexafluorophosphate N-oxide; Cl-Trt for chlorotrityl resin; ESI-MS for Electrospray ionization mass spectroscopy; IR for infrared spectroscopy; HPLC for high performance liquid chromatography; t_{R} for retention time; NMR for nuclear magnetic resonance; LG for leaving group; PG for Protecting Group; and NMP for N-methylpyrrolidone.

The following Schemes and Examples are provided for the purposes of illustration and are not intended to limit the scope of the present invention. The invention is not limited in scope by the exemplified embodiments, which are intended as illustrations of individual aspects of the invention. Various modifications of the invention in addition to those shown and described herein will become apparent to those skilled in the art from the foregoing description and accompanying drawings. Such modifications are intended to fall within the scope of the appended claims.

Preparation of Compounds of the Invention

Polystyrene and polyethylenglycol grafted to polystyrene are among the compounds which can be used as polymeric supports. These supports include an acid-labile linker such as XAL(((9-(amino)xanthen-2-yl)oxy)butanoic acid handle), and Rink (p-((R,S)-α-(1-(9H-fluoren-9-yl)-methoxyformamido)-2,4-dimethoxybenzyl)-phenoxyacetic acid).

Fmoc-Rink linker and solid supports were supplied by Calbiochem-Novabiochem AG. DIPCDI was obtained from Fluka Chemika (Buchs, Switzerland) and HOBt from Albatross Chem. Inc. (Montreal, Canada.) Solvents for peptide synthesis and RP-HPLC were obtained from SDS (Barcelona, Spain). Trifluoroacetic acid was supplied by KaliChemi (Bad Wimpfen, Germany). Semicarbazide hydrochloride, hydrogen peroxide, horseradish perixidase and other chemicals were purchased from Sigma Aldrich (St. Louis, Mo., USA). Purified human VAP-1 was a kind gift from BioTie Therapeutics (Turku, Finland). Amplex red reagent (10-acetyl-3,7-dihydroxyphenoxazine) was from Molecular Probes (Eugene, Oreg., USA). Other chemicals were obtained from Aldrich (Milwaukee, Wis.) and were of the highest purity grade available. All commercial reagents and sovents were used as received. HPLC was performed using an Alliance 2795 Waters Chromatography system with a reverse-phase column C₁₈X-Terra 5 μm 4.6×100 mm with UV detection at 220 and 254 nm. Mass spectra were recorded on a Waters Alliance HT 2795 system with Dual λ Absorbance detector 2487 and Mocromass ZQ Mass Spectrometer. IR were performed by Thermo Nicolet FT-IR Nexus spectrometer 4000-400 cm⁻¹ range. Solid-phase reactions were performed in polypropylene syringes fitted with a polyethylene porous disc. Solvents and soluble reagents were removed by filtration. The purity of the aryalkylamines synthesized was determined by HPC using a C₁₈X-Terra 5 μm 4.6×100 mm column with linear gradient 0% B-100% B in 10 min (A: 0.1 TFA % in H₂O, B: 0.1 TFA % in CAN, 1 mL/min) with Uv detection at 220/254 nm. All compounds were characterized by HPLC-MS.

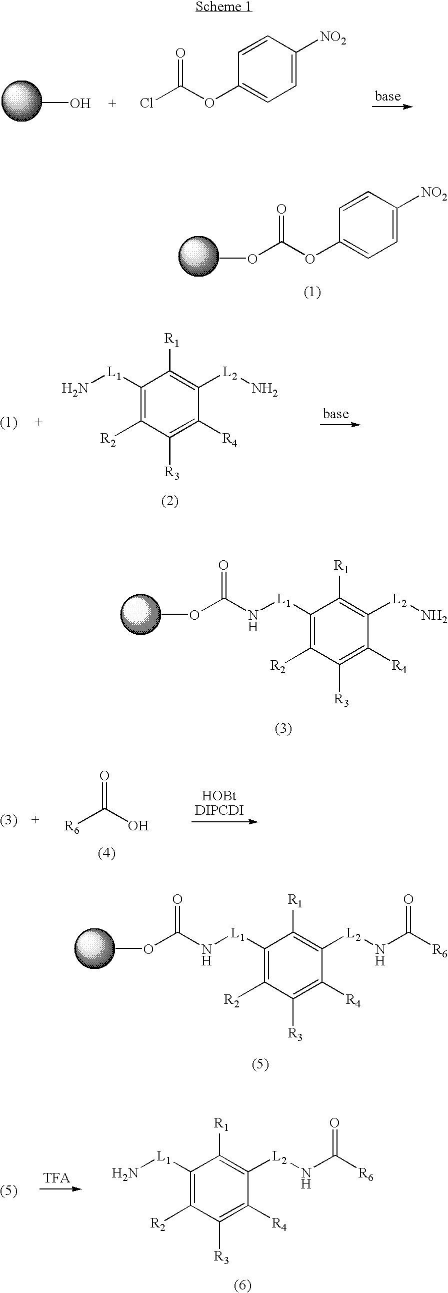

Compounds of formula (6), wherein L₁, L₂, R₁, R₂, R₃, R₄, and R₆ are as defined in Formula (I), are prepared as described in Scheme 1. A hydroxy resin (polymeric support) is treated with 4-nitrophenyl chloroformate and a base such as diisopropylethylamine in an appropriate solvent to provide carbonates of formula (1). A carbonate of formula (1) is treated with a base, such as triethylamine, and a compound of formula (2), purchased commercially or prepared using methods well known in the art, in an appropriate solvent to provide carbamates of formula (3). A carbamate of formula (3) is treated with an acid of formula (4), HOBt, and DIPCDI in an appropriate solvent to provide compounds of formula (5). A compound of formula (5) is treated with trifluoroacetic acid in an appropriate solvent to provide compounds of formula (6).

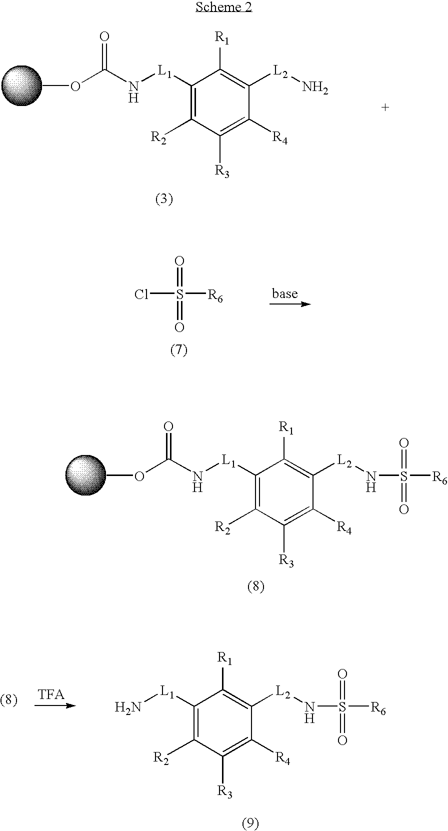

Compounds of formula (9), wherein L₁, L₂, R₁, R₂, R₃, R₄, and R₆ are as defined in Formula (I), are prepared as described in Scheme 2. A compound of formula (3) is treated with a sulfonyl chloride of formula (7) and a base, such as diisopropylethylamine, in an appropriate solvent to provide compounds of formula (8). A compound of formula (8) is treated with trifluoroacetic acid in an appropriate solvent, such as DCM, to provide compounds of formula (9).

### N-(3-Aminomethyl-benzyl)-carbamate resin

Wang resin (1 g, 1.1 mmol/gra d) was treated with 4-nitrophenyl chloroformate (5 eq., 0.97 g) and DIEA (5 eq., 57 μL) in NMP (10 mL) overnight at 60° C. The resin was then washed with NMP (5×1), DMF (5×1) and DCM (5×1). The corresponding 4-nitrophenylcarbonate resin was reacted with 1,3-bis(aminomethyl)benzene (5 eq., 59 μL) and DIEA (15 eq., 2.32 mL) in DCM in 10 mL DCM overnight at room temperature. The resin was then washed with DMF (5×1) and DCM (5×1) to eliminate excess amine. The reaction was followed by IR and Kaiser (ninhydrine test) test, Kaiser, E. et al., Anal. biochem. (1970) 34 page 594.

### General Method for the Synthesis of N-(3-aminomethyl-benzyl)-sulfonamide Derivatives

N-(3-Aminomethyl-benzyl)-carbamate resin (100 mg, 1.1 mmol/gr) was reacted with 5 equivalents of the corresponding sulfonyl chloride derivative (R₆SO₂Cl.) and DIEA (5 eq.) in DCM overnight. The resin was filtered and washed with DCM (5×1 min), and the course of the reaction was evaluated by the ninhydrine test. The N-(3-aminomethyl-benzyl)-sulfonamide derivative resin was cleaved from the resin with TFA-DCM (95:5) for 2 h at rt. The solution was filtered off and evaporated to dryness under low pressure. The corresponding N-(3-aminomethyl-benzyl)-sulfonamide derivative was analyzed by HPLC-MS, 220 nm.

### General Method for the Synthesis of N-(3-aminomethyl-benzyl)-acylamide Derivatives

N-(3-Aminomethyl-benzyl)-carbamate resin (100 mg, 1.1 mmol/gr) was reacted for 2 h with R₆COOH/HOBt/DIPCDI (3 eq.:3 eq.:3 eq.) as acylating mixture in DMF for 2 h at rt. The resin was filtered and washed with DMF (5×1 min) and DCM (5×1 min), and the course of the reaction was followed by the Kaiser test. The N-(3-aminomethyl-benzyl)-acetamide derivative carbamate-resin was cleaved with TFA-DCM (95:5) for 2 h at rt. The solution was filtered off and evaporated under low pressure to dryness. The corresponding N-(3-aminomethyl-benzyl)-acylamide derivatives were analyzed by HPLC-MS, which showed that the purity attained was over 75% in all cases.

Examples 2-12, shown below in Table 1, were prepared essentially according to the synthetic methodology described above.

TABLE 1

| | | | | | Human | Mouse |
|---|---|---|---|---|---|---|
| | | | | | SSAO/VAP-1 | SSAO/VAP-1 |
| | | | | | activity | activity |
| | Compound | | | | % relative to | % relative to |
| Example | Name | Purity (%) | MW | MS | benzylamine^{b} | benzylamine^{b} |
| 1 | benzylamine | — | — | — | 100 | 100 |
| 2 | N-(3-(amino- | 87 | 178.2 | 179.1 | 3 ± 1 | 16 ± 4 |
| | methyl)benzyl) | | | | | |
| | acetamide | | | | | |
| 3 | N-(3-(amino- | 88 | 192.2 | 129.9 | 4 ± 1 | 27 ± 1 |
| | methyl)benzyl) | | | | | |
| | propionamide | | | | | |
| 4 | N-(3-(amino- | 87 | 304.1 | 304.7 | 38 ± 3 | 61 ± 5 |
| | methyl)benzyl)- | | | | | |
| | 2-iodoacetamide | | | | | |
| 5 | allyl 3-(amino- | 99 | 220.2 | 220.8 | 21 ± 1 | 49 ± 3 |
| | methyl)benzyl | | | | | |
| | carbamate | | | | | |
| 6 | N-(3-(amino- | 99 | 240.3 | 240.8 | 17 ± 1 | 58 ± 4 |
| | methyl)benzyl) | | | | | |
| | benzamide | | | | | |
| 7 | (S)-2- | 97 | 311.3 | 343.9 | 2 ± 1 | 13 ± 3 |
| | acetamido-N- | | | | | |
| | (3-(amino- | | | | | |
| | methyl)benzyl)- | | | | | |
| | 2-phenyl- | | | | | |
| | acetamide | | | | | |
| 8 | N-(3-(amino- | 80 | 290.3 | 290.9 | 12 ± 2 | 14 ± 3 |
| | methyl)benzyl)- | | | | | |
| | 4-methyl- | | | | | |
| | benzene- | | | | | |
| | sulfonamide | | | | | |
| 10 | N-(3-(amino- | 85 | 256.1 | 256.9 | 5 ± 1 | 26 ± 1 |
| | methyl)benzyl)- | | | | | |
| | 4-hydroxy- | | | | | |
| | benzamide | | | | | |
| 11 | 4-(amino- | 99 | 269.3 | 269.9 | 17 ± 2 | 12 ± 1 |
| | methyl)-N-(3- | | | | | |
| | (amino- | | | | | |
| | methyl)benzyl) | | | | | |
| | benzamide | | | | | |
| 12 | N-(3-(amino- | 93 | 319.2 | 320.8 | 16 ± 2 | 46 ± 1 |
| | methyl)benzyl)- | | | | | |
| | 4-bromobenzamide | | | | | |

^{b}Human and mouse SSAO activity were determined by detecting the production of hydrogen peroxide in the presence of the different compounds present at 1 mM for human and 100 μM for mice activity measurements.

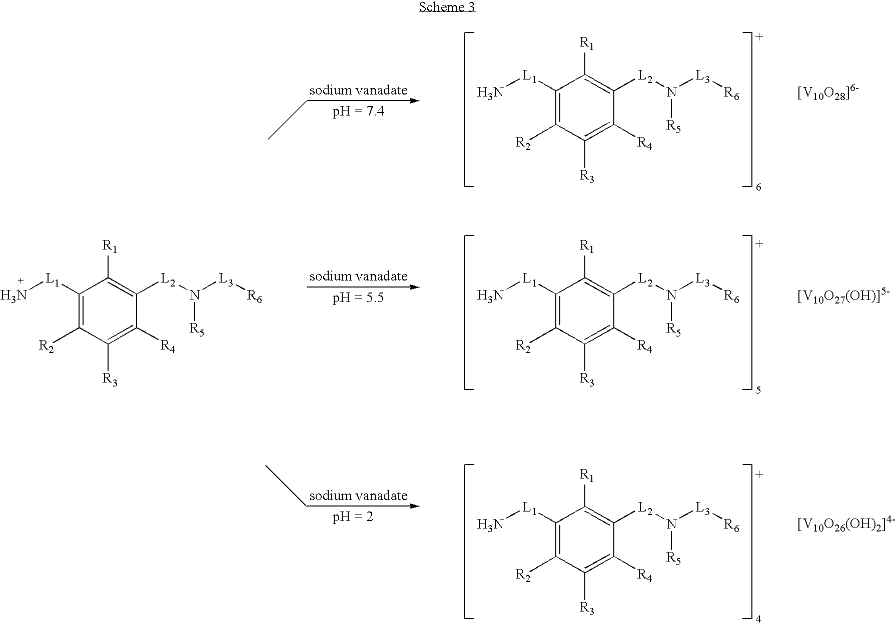

Hexaquis, pentaquis, and tetraquis decavanadate compounds of Formula (I), wherein L₁, L₂, L₃, R₁, R₂, R₃, R₄, and R₆ of the ammonium ion are as defined in Formula (I), are prepared as described in Scheme 3: Sodium vanadate in water is treated with an acid, such as HCl, to pH=7.4 followed by addition of the ammonium analog to provide [hexaquis(ammonium)]₆ [V₁₀O₂₈]⁶⁻ salts of Formula (I). An essentially similar procedure, at pH=5.5 and 2, is used to prepare [pentaquis(ammonium)]₅ [V₁₀O₂₈]⁵⁻ and [tetraquis(ammonium)]₄ [V₁₀O₂₈]⁴⁻ salts of Formula (I), respectively.

Using the synthetic methodology described herein, the following compounds of Formula (I) can be prepared:
[{3-[(propionylamino)methyl]phenyl}methanammonium]₆[V₁₀O₂₈];
[(3-{[(iodoacetyl)amino]methyl}phenyl)methanammonium]₆[V₁₀O₂₈];
[[3-({[(allyloxy)carbonyl]amino}methyl)phenyl]methanammonium]₆[V₁₀O₂₈];
[{3-[(benzoylamino)methyl]phenyl}methanammonium]₆[V₁₀O₂₈];
[[3-({[(2S)-2-(acetylamino)-2-phenylacetyl]amino}methyl)phenyl]methanammonium]₆ [V₁₀O₂₈];
[[3-({[(2R)-2-(acetylamino)-2-phenylacetyl]amino}methyl)phenyl]methanammonium]₆[V₁₀O₂₈];
[(3-{[(4-hydroxybenzoyl)amino]methyl}phenyl)methanammonium]₆[V₁₀O₂₈];
[[3-({[4-(aminomethyl)benzoyl]amino}methyl)phenyl]methanammonium]₆[V₁₀O₂₈];
[(3-{[(4-bromobenzoyl)amino]methyl}phenyl)methanammonium]₆[V₁₀O₂₈];
[[3-({[(4-methylphenyl)sulfonyl]amino}methyl)phenyl]methanammonium]₆[V₁₀O₂₈];
[{3-[(propionylamino)methyl]phenyl}methanammonium]₅[V₁₀O₂₇OH];
[(3-{[(iodoacetyl)amino]methyl}phenyl)methanammonium]₅[V₁₀O₂₇OH];
[[3-({[(allyloxy)carbonyl]amino}methyl)phenyl]methanammonium]₅[V₁₀O₂₇OH];
[{3-[(benzoylamino)methyl]phenyl}methanammonium]₅[V₁₀O₂₇OH];
[[3-({[(2S)-2-(acetylamino)-2-phenylacetyl]amino}methyl)phenyl]methanammonium]₅[V₁₀O₂₇OH];
[[3-({[(2R)-2-(acetylamino)-2-phenylacetyl]amino}methyl)phenyl]methanammonium]₅[V₁₀O₂₇OH];
[(3-{[(4-hydroxybenzoyl)amino]methyl}phenyl)methanammonium]₅[V₁₀O₂₇OH];
[[3-({[4-(aminomethyl)benzoyl]amino}methyl)phenyl]methanammonium]₅[V₁₀O₂₇OH];
[(3-{[(4-bromobenzoyl)amino]methyl}phenyl)methanammonium]₅[V₁₀O₂₇OH];
[[3-({[(4-methylphenyl)sulfonyl]amino}methyl)phenyl]methanammonium]₅[V₁₀O₂₇OH];
[{3-[(propionylamino)methyl]phenyl}methanammonium]₄[V₁₀O₂₆(OH)₂];
[(3-{[(iodoacetyl)amino]methyl}phenyl)methanammonium]₄[V₁₀O₂₆(OH)₂];
[[3-({[(allyloxy)carbonyl]amino}methyl)phenyl]methanammonium]₄[V₁₀O₂₆(OH)₂];
[{3-[(benzoylamino)methyl]phenyl}methanammonium]₄[V₁₀O₂₆(OH)₂];
[[3-({[(2S)-2-(acetylamino)-2-phenylacetyl]amino}methyl)phenyl]methanammonium]₄[V₁₀O₂₆(OH)₂];
[[3-({[(2R)-2-(acetylamino)-2-phenylacetyl]amino}methyl)phenyl]methanammonium]₄[V₁₀O₂₆(OH)₂];
[(3-{[(4-hydroxybenzoyl)amino]methyl}phenyl)methanammonium]₄[V₁₀O₂₆(OH)₂];
[[3-({[4-(aminomethyl)benzoyl]amino}methyl)phenyl]methanammonium]₄[V₁₀O₂₆(OH)₂];
[(3-{[(4-bromobenzoyl)amino]methyl}phenyl)methanammonium]₄[V₁₀O₂₆(OH)₂];
and
[[3-({[(4-methylphenyl)sulfonyl]amino}methyl)phenyl]methanammonium]₄[V₁₀O₂₆(OH)₂].

Further, using the synthetic methodology described herein, the following compounds of Formula (II) can be prepared:
N-(3-(aminomethyl)benzyl)propionamide;
N-(3-(aminomethyl)benzyl)-2-iodoacetamide;
allyl 3-(aminomethyl)benzylcarbamate;
N-(3-(aminomethyl)benzyl)benzamide;
(S)-2-acetamido-N-(3-(aminomethyl)benzyl)-2-phenylacetamide;
N-(3-(aminomethyl)benzyl)-4-methylbenzenesulfonamide;
N-(3-(aminomethyl)benzyl)-4-hydroxybenzamide;
4-(aminomethyl)-N-(3-(aminomethyl)benzyl)benzamide; and
N-(3-(aminomethyl)benzyl)-4-bromobenzamide.

### Effects of Hexaquis(benzylammonium) decavanadate, Pentaquis(benzylammonium) decavanadate and Tetraquis(benzylammonium) decavanadate on Glucose Transport in Isolated Adipocytes

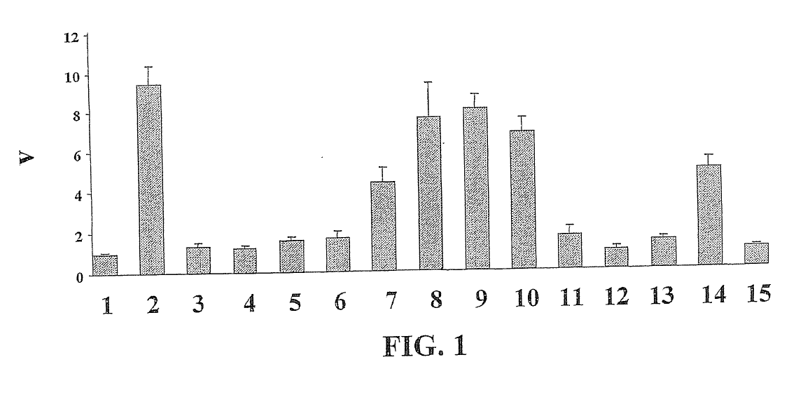

As shown in FIG. 1, hexaquis(benzylammonium) decavanadate induced the stimulation of glucose transport which was perceptible from concentrations of 0.5 μM, with a maximal effect observed 2.5 μM and the semimaximal effect above 1 μM.

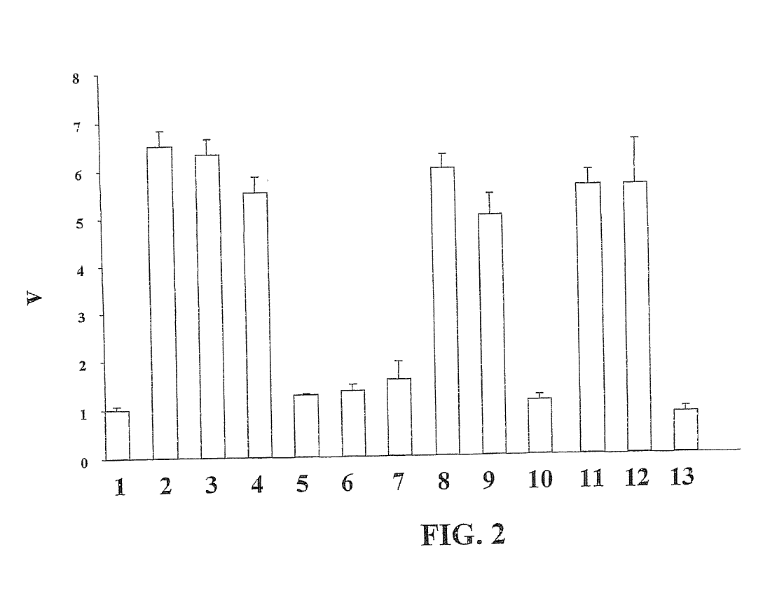

The stimulatory effect of the hexaquis(benzylammonium) decavanadate was completely blocked by semicarbazide, which indicates that the semicarbazide-sensitive amine oxidase activity is required for the effect. The maximum effect provoked by incubation with hexaquis(benzylammonium) decavanadate was greater than that produced by the presence of benzylamine and vanadate in combination (FIG. 1). These results indicated that hexaquis(benzylammonium) decavanadate is an insulin mimetic agent more powerful than the combination of vanadate and benzylamine. In similar assays, the activity of hexaquis(benzylammonium) decavanadate, pentaquis(benzylammonium) decavanadate and tetraquis(benzylammonium) decavanadate on glucose transport activity in isolated rat adipocytes were analyzed. The three compounds caused a pronounced stimulation on glucose transport (FIG. 2) and in the presence of the semicarbazide inhibitor this effect was inhibited.

### Effect of the Chronic Administration of Hexaquis(benzylammonium) decavanadate in Diabetic Rats

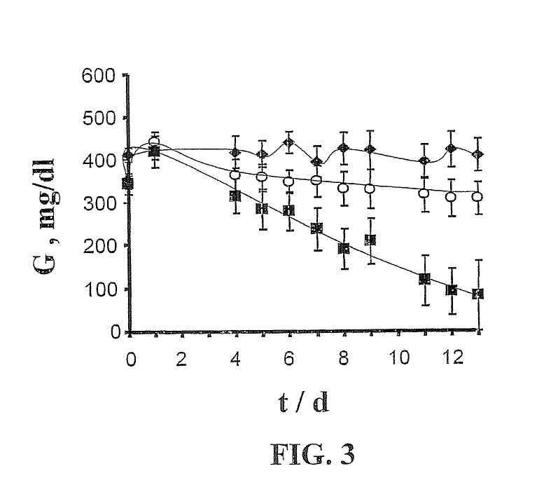

The effect of chronic administration of hexaquis(benzylammonium) decavanadate on glycemia from diabetic rats was determined. Diabetes was induced in rats by intravenous administration of streptozotocin, which destroys the β-pancreatic cells that produce insulin. Treated rats with buffered solution used as solvent or with sodium decavanadate, did not modify substantially its glycemia during the two weeks of treatment (FIG. 3). Under these conditions, administration to the rats of hexaquis(benzylammonium) decavanadate produced a rapid reduction of the hyperglycemia that was detected after only four days of treatment (FIG. 3). After eleven days of treatment, glycemia in hexaquis(benzylammonium) decavanadate-treated rats was similar to the non-diabetic rats. At fourteen days of treatment, adipocytes from chronically hexaquis(benzylammonium) decavanadate-treated rats were isolated and glucose transport velocity determined; adipocytes of hexaquis(benzylammonium) decavanadate-treated rats showed an increased glucose transport under basal conditions equivalent to that seen in the presence of insulin. Moreover, an inverse correlation was detected between animal glycemia and basal glucose transport velocity, which suggested that adipocytes played a role in the antidiabetic effects of hexaquis(benzyl-ammonium) decavanadate.

### Effect of Oral and Chronic Administration of Hexaquis(benzylammonium) decavanadate in Diabetic Rats

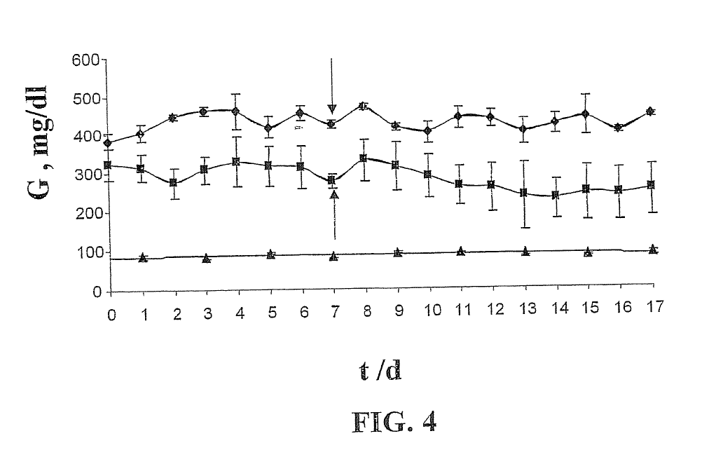

Diabetes was induced in rats by intravenous administration of streptozotocin, and subsequently, a hexaquis(benzylammonium) decavanadate or sodium decavanadate unique dose was administered to the rats. Glycemia was not affected substantially in sodium decavanadate-treated rats during the seventeen days of treatment (FIG. 4). Under these conditions, administration of a 5 μmol/kg/day dose of hexaquis(benzylammonium) decavanadate for seven days produced a moderate decrease of hyperglycemia that was detected after but two days of treatment (FIG. 4). After seven days of treatment, the dose was increased at 10 μmol/kg/day which was maintained for an additional ten days. The dosage increase produced an additional decrease in glycemia of the animals. Thus, glycemia in sodium decavanadate treated rats was approximately 450 mg/dl and glycemia of hexaquis(benzylammonium) decavanadate treated rats was approximately 250 mg/dl.

### Analysis of Insulin Mimetic Capacity of Certain Vanadium Arylalkylamines

Adipose cells from Wistar rats were incubated for 45 minutes in basal conditions (Basal) or in the presence of 100 nM insulin (Ins) and different concentrations of. hexaquis(benzylammonium) decavanadate (B6V10) in the absence or in the presence of 1 mM semicarbazide (SCZ). Subsequently, 2-DG transport was measured over a 5 min. interval.

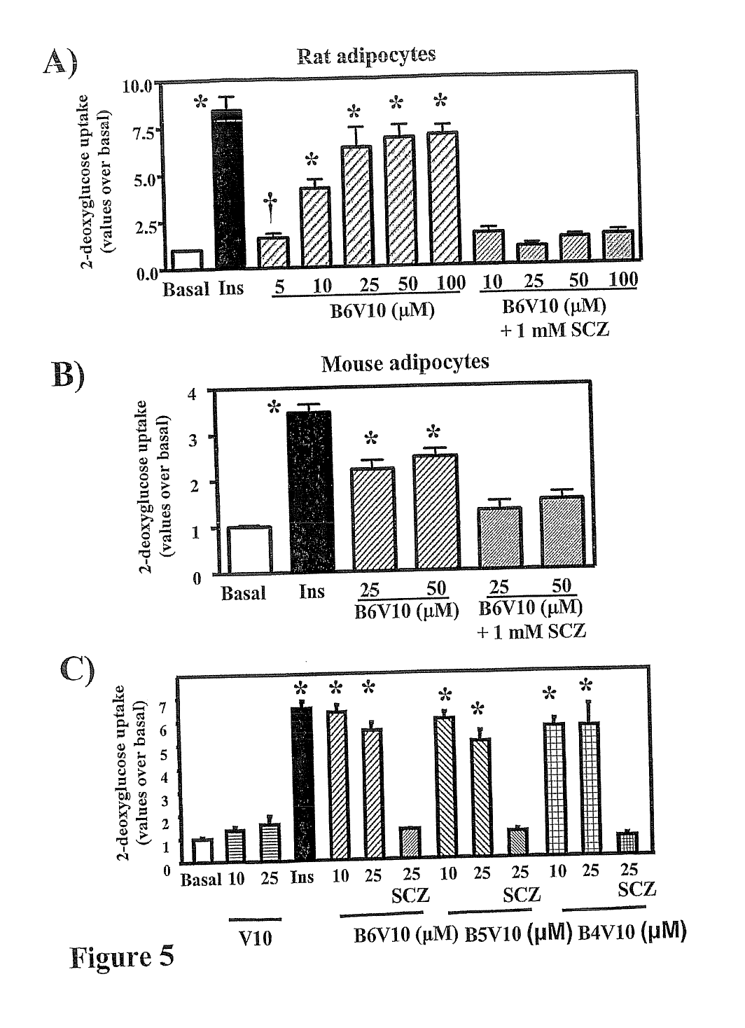

The results of these experiments are shown in FIG. 5A through 5C. B6V10 stimulated glucose transport in rat adipocytes in a concentration-dependent manner (FIG. 5A) and the maximal effect was 85% of the maximal stimulation caused by insulin. Notably, 25 μM B6V10 showed a greater stimulation of glucose transport than the combination of 100 μM benzylamine and 100 μM vanadate (data not shown). The stimulatory effect of B6V10 was completely blocked by semicarbazide, which indicates that SSAO activity is required to observe the effect of B6V10 in these cells. In contrast, sodium decavanadate salt (V10) alone at concentrations ranging from 5 to 50 μM did not stimulate glucose transport (data not shown; see FIG. 5C).

Similar stimulatory effects of B6V10 were detected in isolated mouse adipocytes (FIG. 5B). Adipose cells from FVB mice were incubated for 45 minutes in basal conditions (Basal) or in the presence of 100 nM insulin (Ins), and different concentrations of hexaquis(benzylammonium) decavanadate (B6V10) in the absence or in the presence of 1 mM semicarbazide (SCZ) and thereafter, 2-DG transport was measured over 5 min.

The addition of benzylamine and V₁₀ at equivalent concentrations showed no effect on glucose transport in isolated mouse adipocytes (data not shown), and stimulation of glucose transport by 100 μM B6V10 (93% increase) was greater than the stimulation that resulted from the combination of 1 mM benzylamine and 1 mM vanadate (51% increase). This result suggested that B6V10 has additional relevant biological properties compared to their combined components.

The effects of the three tested compounds (B6V10, B5V10 and B4V10) were compared. Adipose cells from Wistar rats were incubated for 45 minutes in basal conditions (Basal) or in the presence of 100 nM insulin (Ins), and different concentrations of decavanadate (V10), hexaquis(benzylammonium) decavanadate (B6V10), pentaquis(benzylammonium) decavanadate (B5V10) or tetraquis(benzylammonium) decavanadate (B4V10) in the absence or in the presence of 1 mM semicarbazide (SCZ). 2-DG transport was measured over 5 min. intervals. All three compounds showed a similar potency as activators of glucose transport activity in isolated rat adipocytes (FIG. 5C). The stimulation of all three compounds on glucose transport was blocked in the presence of semicarbazide. These results indicated that a lower ratio benzylamine/vanadium does not alter the insulin replacement potency of the arylalkylamine vanadium salts.

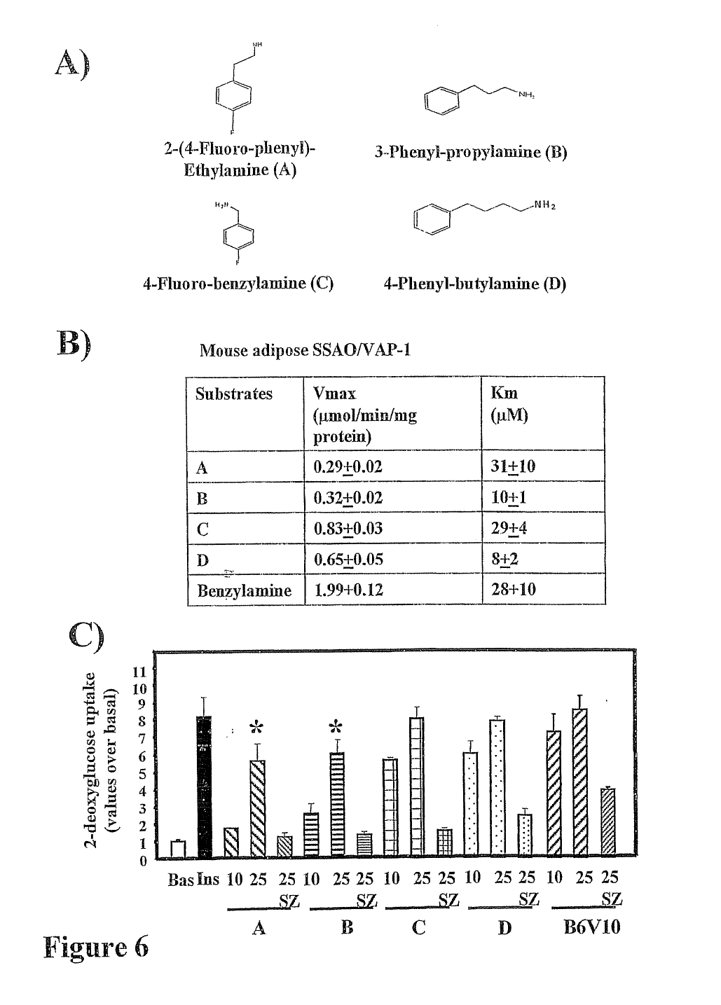

In additional experiments, compounds shown in FIG. 6A, 2-(4-fluoro-phenyl)ethylamine (compound A), 3-phenylpropylamine (compound B), 4-fluoro-benzylamine (compound C) and 4-phenylbutylamine (compound D) were assessed using the experimental methods set forth above for the capacity to stimulate 2-DG uptake in isolated rat adipocytes. These results are shown in FIG. 6B. Adipose cells from Wistar rats were incubated for 45 minutes in basal conditions (Basal) or in the presence of 100 nM insulin (Ins), and different concentrations of vanadium salts of 2-(4-fluoro-phenyl)-ethylamine (compound A), 3-phenyl-propylamine (compound B), 4-fluoro-benzylamine (compound C) and 4-phenyl-butylamine (compound D). 2-DG transport was measured over 5 min. All four compounds markedly stimulated glucose transport of rat adipocytes.

TABLE 2

SSAO activity
(% relative to benzylamine^{a})

| Compound | Human | Mouse |
|---|---|---|
| 4-fluorobenzylamine | 31.70 | 77.20 |
| 3-phenylpropylamine | 60.30 | 44.80 |
| 4-phenylbutylamine | 147.63 | 61.18 |
| 2-(4-fluoro-phenyl)ethylamine | 16.30 | 59.80 |

### Materials

2-[1,2-³H]-D-deoxyglucose (26 Ci/mmol) was obtained from PerkinElmer Life and Analytical Sciences Products (Boston, Mass.) and [¹⁴C]Benzylamine (59 Ci/mmol) was obtained from Amersham Biosciences (Little Chalfont, Buckinghamshire, England). Purified porcine insulin was a kind gift from Eli Lilly (Indianapolis, Ind.). Semicarbazide hydrochloride, benzylamine hydrochloride, sodium orthovanadate, wortmannin and other chemicals were purchased from Sigma Aldrich (St. Louis, Mo.). LY294002 was purchased from Calbiochem (San Diego, Calif.). Ketamine was obtained from Merieux (Imalgene, Merieux, France). Collagenase type I was obtained from Worthington (Lakewood, N.J.) and collagenase P from Roche Diagnostics (Basel, Switzerland). The osmotic minipumps used in chronic studies were from Alza Corporation (Palo Alto, Calif.). All electrophoresis reagents and molecular weight markers were obtained from Bio-Rad. Enhanced chemiluminescence reagents (super signal substrate) were from Amersham (Arlington Heights, Ill.). Anti-phospho-tyrosine monoclonal antibody and anti-insulin receptor β-chain polyclonal antibodies were purchased from BD Biosciences (Franklin Lakes, N.J.). Anti-phospho-Thr308-PKB and anti phosho-Ser473-PKB polyclonal antibodies were purchased from Cell Signaling Technologies (Beverly, Mass.).

Fmoc-Rink linker and solid supports were supplied by Calbiochem-Novabiochem AG. DIPCDI was obtained from Fluka Chemika (Buchs, Switzerland) and HOBt from Albatross Chem. Inc. (Montreal, Canada). Solvents for peptide synthesis and RP-HPLC were obtained from SDS (Barcelona, Spain). Trifluoroacetic acid was supplied by KaliChemie (Bad Wimpfen, Germany). Semicarbazide hydrochloride, benzylamine hydrochloride, hydrogen peroxide, horseradish peroxidase and other chemicals were purchased from Sigma Aldrich (St. Louis, Mo., USA). Purified human VAP-1 was a kind gift from BioTie Therapeutics (Turku, Finland). Amplex red reagent (10-acetyl-3,7-dihydroxyphenoxazine) was from Molecular Probes (Eugene, Oreg., USA). Other chemicals were obtained from Aldrich (Milwaukee, Wis.) and were of the highest purity grade available. All commercial reagents and solvents were used as received. HPLC was performed using an Alliance 2795 Waters Chromatography system with a reverse-phase column C₁₈X-Terra 5 μm 4.6×100 mm with UV detection at 220 and 254 nm. Mass spectra were recorded on a Waters Alliance HT 2795 system with Dual [symbol] Absorbance detector 2487 and Micromass ZQ Mass Spectrometer. IR were performed by Thermo Nicolet FT-IR Nexus spectrometer 4000-400 cm⁻¹ range. Solid-phase reactions were performed in polypropylene syringes fitted with a polyethylene porous disc. Solvents and soluble reagents were removed by filtration. Solvents and soluble reagents were removed by filtration. The purity of the arylalkylamines synthesized was determined by HPLC using a C₁₈X-Terra 5 μm 4.6×100 mm column with linear gradient 0% B-100% B in 10 min (A: 0.1 TFA % in H₂O, B: 0.1 TFA % in ACN, 1 mL/min) with UV detection at 220/254 nm. All compounds were characterized by HPLC-MS.

### Animals

Male Wistar rats weighting 180-220 g were purchased from Harlan (Interfauna Ibérica S.A., Spain). Diabetes was induced by a single intraperitoneal injection of a freshly prepared solution of streptozotocin (in some studies the dose was 45 mg/kg body weight and in some others 100 mg/kg body weight dissolved in 50 mM citrate buffer, pH 4.5). Only diabetic animals with glycemia above 300 mg/dl were used. The animals were housed in animal quarters at 22° C. with a 12 h light/12 h dark cycle and were fed ad libitum. All procedures used were approved by the animal ethical committee of the University of Barcelona, Spain. Male mice C57 BL/Ks bearing the db/db mutation (Jackson Laboratories, Bar Harbor, Me.) were purchased from Harlan France (Gannat, France). C57BL/6J male mice were assigned for 16 weeks to very high-fat diet containing (in kcal): 72% from fat, 28% from proteins and <1% from carbohydrates (Burcelin et al., 2002, Am. J. Physiol. Endocrinol. Metab 282: E834-E842).

### Chronic Treatments of Diabetic Animals

Osmotic minipumps delivering B6V10 (2.5 μmol/kg body wt/day) or decavanadate (2.5 μmol/kg body wt/day) were implanted subcutaneously in diabetic rats anaesthetised by ketamine hydrochloride (95 mg/kg) and xylasine (10 mg/kg). Animals that did not receive B6V10 or decavanadate were sham-operated. Glycemia was measured on arterio-venous blood collected from the tail vessels at 09:00 am for two weeks, before the administration of vanadate. Insulin concentrations were determined before and after treatment. In another set of experiments, B6V10 was orally administered at a single dose of 5 μmol/kg/day during the first week and 10 μmol/kg/day during 2 additional weeks by gastric gavage. A control group received the corresponding decavanadate salt in the absence of benzylamine. At the end of the treatment, animals were sacrificed and the liver, fat pad, heart and lung were kept at −80° C. and the plasma at −20° C. until their use for in vitro analysis.

### Amine Oxidase Activity Assays

The continuous spectrophotometric detection of SSAO-dependent H₂O₂ production based on a peroxidase-coupled reaction was performed as previously described by Abella et al. (2004, Diabetologia 47: 429-438) and following the procedure described by Holt et al., (1997, Anal. Biochem. 244: 384-392).

### Analytical Methods

In glucose tolerance tests and in chronic treatments, circulating glucose concentrations were determined by a rapid glucose analyser (Accutrend® Sensor Comfort, Roche, Basel. Switzerland). Plasma insulin (IRI) concentration was determined by ELISA method using a kit obtained from Crystal Chem. Inc. (Downers Grove, Ill.). Plasma triglycerides (Biosystems, Barcelona, Spain) and NEFAS (Wako Chemicals, Neuss, Germany) were determined with standard calorimetric methods.

### Analysis of Intracellular Signaling

Isolated fat cells were disrupted for total membrane preparation by hypo-osmotic lysis in a 20 mM HES buffer and an antiprotease and antiphosphatase cocktail as reported by Abella et al. (2003, Diabetes 52: 1004-1013). Protein concentrations were determined by the Bradford method (Bradford, 1976, Anal. Biochem. 72: 248-254) with gamma-globulin as protein concentration standard. Immunoprecipitation and immunoblot assays were performed as previously described by Abella et al. (2004, Diabetologia 47: 429-438) with the use of a monoclonal antiphosphotyrosine antibody for the immunoprecipitation and an anti-insulin receptor antibody for immunobloting, respectively. SDS-polyacrylamide gel electrophoresis was performed on membrane proteins following conventional procedures. Proteins were transferred to Immobilon and immunoblotting was performed as reported by Castello et al. (1994, J. Biol. Chem. 269: 5905-5912).

### Calculations and Statistical Analysis

Insulin and glucose responses during the glucose tolerance test were calculated as the incremental plasma values integrated over a period of 120 min after injection of glucose. Areas under curves of insulin and glucose responses were calculated using the Graph Prism program (Graphpad Software, Inc., San Diego, Calif.). Data were presented as mean ±SEM and unpaired Student's t test was used to compare two groups. When experimental series involved more than two groups, statistical analysis was done by one-way or two-way ANOVA and further post-hoc (Dunnett, Tukey or Bonferroni) t tests. Statistical analysis was performed with SPSS 11.0 or GraphPad Prism 4 programs.

### Mechanism of Action of Vanadium Arylalkylamines

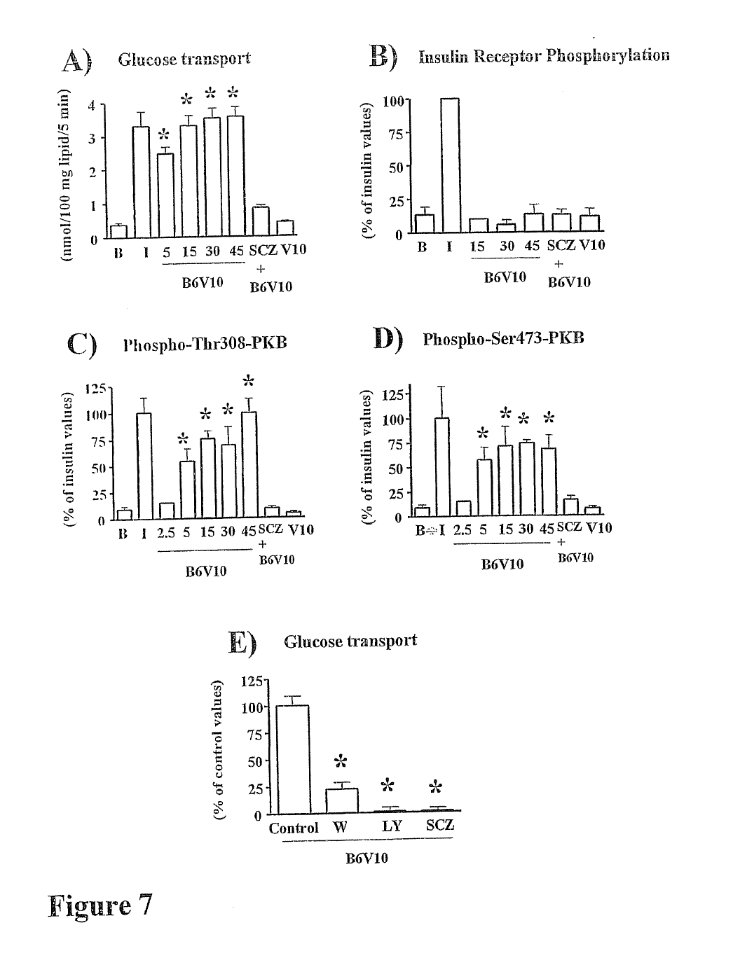

The mechanism of action of B6V10 was investigated in isolated rat adipocytes. Adipose cells from Wistar rats were incubated for different times in the presence of 25 μM hexaquis(benzylammonium) decavanadate (B6V10). Cells were also incubated in the presence of insulin (100 nM, 45 min), decavanadate (25 μM, 45 min) or semicarbazide (1 mM, 45 min). Subsequently, 2-deoxyglucose uptake (results shown in FIG. 7A), tyrosine phosphorylation of insulin receptor (FIG. 7B), phospho-Thr³⁰⁸-protein kinase B (FIG. 7C) and phospho-Ser⁴⁷³-protein kinase B (FIG. 7D) was measured. B6V10 rapidly stimulated protein kinase B as assessed by the phosphorylation of Thr⁴⁷³ and Ser⁴⁷³ in the rat insulin receptor that was detectable as early as 2.5 min after B6V10 addition (FIG. 7B). The phosphorylation of protein kinase B induced by B6V10 was parallel to activation of glucose transport (FIGS. 7C and 7D). Under these conditions, tyrosine phosphorylation of insulin receptors was undetectable in adipose cells incubated with B6V10, indicating that the initial site of activation of the insulin signalling was downstream from insulin receptor.

The effects of incubation with semicarbazide or phosphatidylinositol 3-kinase inhibitors were also investigated. Adipose cells were incubated with B6V10 (25 μM, 45 min) in the absence or presence of wortmannin (2 μM, 45 min), LY294002 (10 μM, 45 min) or semicarbazide (1 mM, 45 min) and thereafter 2-deoxyglucose uptake was determined during 5 min. Activation of protein kinase B phosphorylation induced by B6V10 was blocked by semicarbazide and it was not observed by decavanadate. In addition, phosphatidylinositol 3-kinase inhibitors wortmannin and LY294002 blocked B6V10-induced glucose transport (FIG. 7E).

### Effectiveness of B6V10 on Glucose Tolerance In Vivo

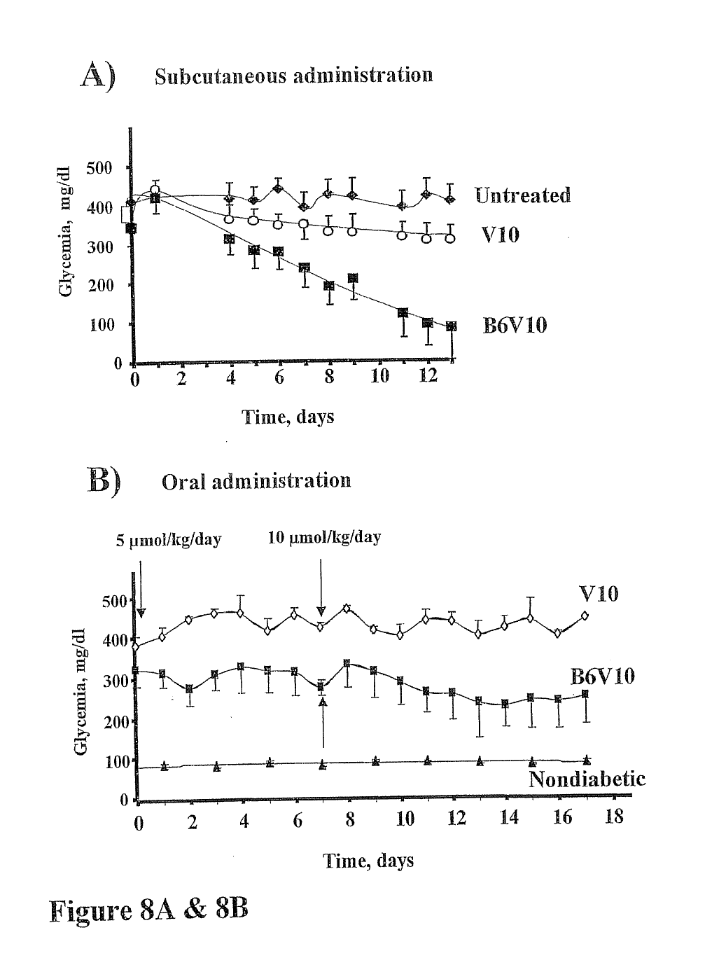

Chronic in vivo efficacy of B6V10 was evaluated in streptozotocin-induced diabetic rats and in db/db mice. Streptozotocin-induced (45 mg/kg) diabetic rats were subcutaneously treated with hexaquis(benzylammonium) decavanadate (2.5 μmol/kg) (B6V10, solid squares, FIG. 8A) or with decavanadate (2.5 μmol/kg) (V10, open circles, FIG. 8A) delivered subcutaneously by osmotic minipumps implanted in the dorsal region. Diabetic rats were also sham-operated (untreated, solid diamonds, FIG. 8A). Chronic subcutaneous administration of B6V10 for 12 days resulted in significant correction of hyperglycemia in streptozotocin-induced diabetic rats (45 mg/kg of streptozotocin) (FIG. 8A). These experiments were repeated using an oral administration protocol. Streptozotocin-induced (45 mg/kg) diabetic rats were orally treated with hexaquis(benzylammonium) decavanadate (5 μmol/kg from day 0 to day 7 and 10 μmol/kg/day from day 7 to day 17) (B6V10, solid squares, FIG. 8B) or received decavanadate (10 μmol/kg) (V10, open circles, FIG. 8B). Nondiabetic rats were also untreated (solid triangles, FIG. 8B). Daily oral administration of B6V10 for 17 days also resulted in significant correction of hyperglycemia in diabetic rats (45 mg/kg of streptozotocin) (FIG. 8B). Treatment with identical doses of decavanadate (V10) did not alter glycemia in streptozotocin-induced diabetic rats (FIGS. 8A and 8B).

### Insulin Replacement Activity of Vanadium Salts of Arylalkylamines

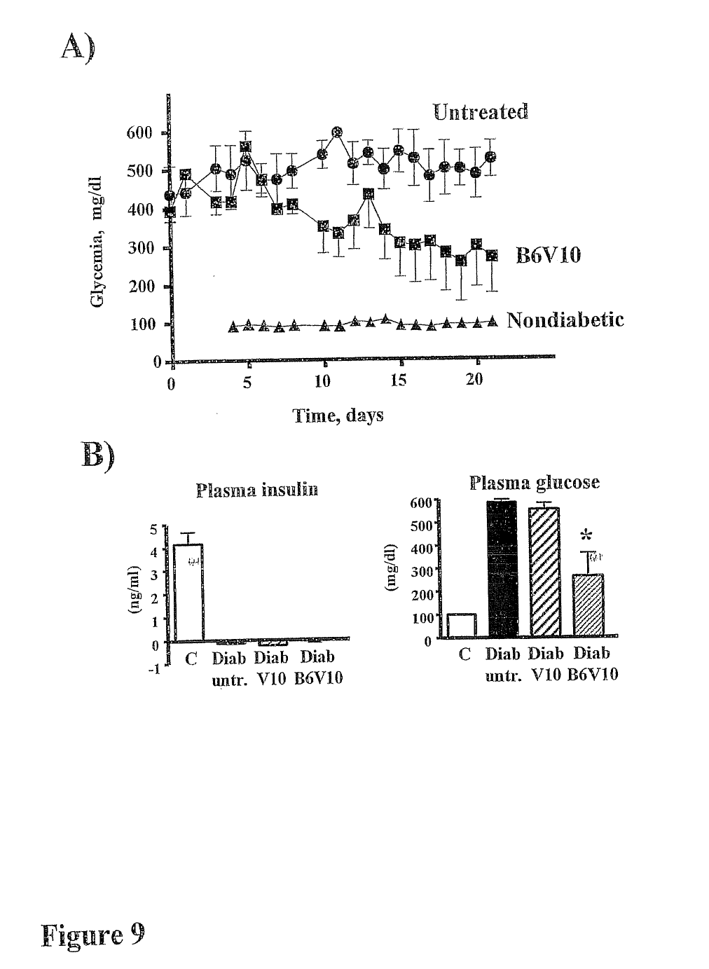

The capacity of B6V10 to exhibit antidiabetic effects in vivo in the complete absence of insulin. To this end, rats were made diabetic by the injection of a large dose of streptozotocin (100 mg/kg) that eliminates β-pancreatic insulin content. These rats showed undetectable levels of insulin in plasma (FIG. 9B). These streptozotocin-induced diabetic rats were subcutaneously treated with B6V10 (2.5 μmol/kg) (solid squares, FIG. 9A) delivered by osmotic minipumps or left untreated (solid circles, FIG. 9A). Sham-operated nondiabetic rats were also untreated (solid triangles, FIG. 9A). Diabetic rats responded to subcutaneous treatment with B6V10 by reducing glycemia (FIG. 9A). However, treatment with decavanadate did not show any change in circulating glucose (data not shown). Chronic treatment with therapeutic doses of B6V10 did not affect body weight or organ weights (data not shown).

The concentration of circulating glucose under these conditions was also assayed. After 28 days of treating diabetic rats with hexaquis(benzylammonium) decavanadate (2.5 μmol/kg) (wide striped bars, FIG. 9B), or with decavanadate (close striped bars, FIG. 9B) delivered by osmotic minipumps, plasma insulin and glucose were measured. Untreated diabetic (solid bars, FIG. 9B) or nondiabetic rats (open bars, FIG. 9B) were similarly assayed as controls. As shown in FIG. 9B, plasma glucose was reduced even without any observable change in the amount of insulin normalized in diabetic rats treated with B6V10 but not with decavanadate alone. These results indicated that B6V10 could be used to replace insulin treatment in human types 1 and 2 diabetes, based in these results in a clinically-accepted animal model of the disease.

It is to be understood that the foregoing describes preferred embodiments of the invention and that modifications may be made therein without departing from the spirit or scope of the invention as set forth in the claims. To particularly point out and distinctly claim the subject matter regarded as invention, the following claims conclude this specification.

We claim:

1. A compound of Formula (I) 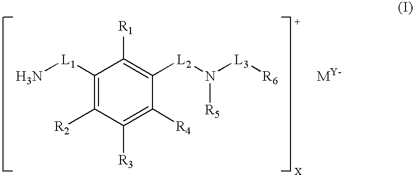
or a pharmaceutically-acceptable salt, solvate, or hydrate thereof, wherein
M is a negatively charged vanadium complex comprising vanadium (“V”) and oxygen, or vanadium, oxygen, and 1 or 2 hydroxy groups;
Y is an integer from 1 to 10;
X is an integer from 1 to 10;
L₁ and L₂ are independently (C₁-C₆)alkylene;
L₃ is —C(O)— or —S(O)₂—;
R₁, R₂, R₃, and R₄ are independently H, (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, or nitro;
R₅ is H or (C₁-C₆)alkyl;
R₆ is (C₁-C₆)alkoxy, (C₂-C₆)alkenyl, (C₂-C₆)alkenyloxy, (C₁-C₆)alkyl, (C₂-C₆)alkynyl, (C₂-C₆)alkynyloxy, aryl, (C₁-C₆)haloalkyl, (C₁-C₆)haloalkoxy, NR₇R₈, —CH(R₉)NR₁₀R₁₁, or —CH₂CH₂NR₁₀R₁₁, wherein the aryl is optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl;
R₇ and R₈ are independently H or (C₁-C₆)alkyl;
R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; and
R₁₀ and R₁₁ are independently H, (C₁-C₆)alkyl, (C₁-C₆)alkylcarbonyl, formyl, or (C₁-C₆)alkoxycarbonyl.

2. The compound according to claim 1 wherein
M is V₁₀O₂₈;
X is 6;
Y is 6;
L₁ is —CH₂—;
L₂ is —CH₂—;
L₃ is —C(O)—;
R₁, R₂, R₃, R₄, and R₅ are H;
R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and
R₇ and R₈ are H.

3. The compound according to claim 1 wherein
M is V₁₀O₂₇OH;
X is 5;
Y is 5;
L₁ is —CH₂—;
L₂ is —CH₂—;
L₃ is —C(O)—;
R₁, R₂, R₃, R₄, and R₅ are H;
R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and
R₇ and R₈ are H.

4. The compound according to claim 1 wherein
M is V₁₀O₂₆(OH)₂;
X is 4;
Y is 4;
L₁ is —CH₂—;
L₂ is —CH₂—;
L₃ is —C(O)—;
R₁, R₂, R₃, R₄, and R₅ are H;
R₆ is (C₂-C₆)alkenyloxy, (C₁-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and
R₇ and R₈ are H.

5. The compound according to claim 1 wherein
M is V₁₀O₂₈;
X is 6;
Y is 6;
L₁ is —CH₂—;
L₂ is —CH₂—;
L₃ is —C(O)—;
R₁, R₂, R₃, R₄, and R₅ are H;
R₆ is —CH(R₉)NR₁₀R₁₁;
R₉ is phenyl;
R₁₀ is —H; and
R₁₁ is (C₁-C₆)alkylcarbonyl.

6. The compound according to claim 1 wherein
M is V₁₀O₂₇OH;
X is 5;
Y is 5;
L₁ is —CH₂—;
L₂ is —CH₂—;
L₃ is —C(O)—;
R₁, R₂, R₃, R₄, and R₅ are H;
R₆ is —CH(R₉)NR₁₀R₁₁;
R₉ is phenyl;
R₁₀ is —H; and
R₁₁ is (C₁-C₆)alkylcarbonyl.

7. The compound according to claim 1 wherein
M is V₁₀O₂₆(OH)₂;
X is 4;
Y is 4;
L₁ is —CH₂—;
L₂ is —CH₂—;
L₃ is —C(O)—;
R₁, R₂, R₃, R₄, and R₅ are H;
R₆ is —CH(R₉)NR₁₀R₁₁;
R₉ is phenyl;
R₁₀ is —H; and
R₁₁ is (C₁-C₆)alkylcarbonyl.

8. The compound according to claim 1 wherein
M is V₁₀O₂₈;
X is 6;
Y is 6;
L₁ is —CH₂—;
L₂ is —CH₂—;
L₃ is —S(O)₂—;
R₁, R₂, R₃, R₄, and R₅ are H; and
R₆ is phenyl optionally substituted with (C₁-C₆)alkyl.

9. The compound according to claim 1 wherein
M is V₁₀O₂₇OH;
X is 5;
Y is 5;
L₁ is —CH₂—;
L₂ is —CH₂—;
L₃ is —S(O)₂—;
R₁, R₂, R₃, R₄, and R₅ are H; and
R₆ is phenyl optionally substituted with (C₁-C₆)alkyl.

10. The compound according to claim 1 wherein
M is V₁₀O₂₆(OH)₂;
X is 4;
Y is 4;
L₁ is —CH₂—;
L₂ is —CH₂—;
L₃ is —S(O)₂—;
R₁, R₂, R₃, R₄, and R₅ are H; and
R₆ is phenyl optionally substituted with (C₁-C₆)alkyl.

11. A compound according to claim 1 that is
[{3-[(propionylamino)methyl]phenyl}methanammonium]₆[V₁₀O₂₈]⁶⁻;
[(3-{[(iodoacetyl)amino]methyl}phenyl)methanammonium]₆[V₁₀O₂₈]⁶⁻;
[[3-({[(allyloxy)carbonyl]amino}methyl)phenyl]methanammonium]₆[V₁₀O₂₈]⁶⁻;
[{3-[(benzoylamino)methyl]phenyl}methanammonium]₆[V₁₀O₂₈]⁶⁻;
[[3-({[(2S)-2-(acetylamino)-2-phenylacetyl]amino}methyl)phenyl]methanammonium]₆[V₁₀O₂₈]⁶⁻;
[[3-({[(2R)-2-(acetylamino)-2-phenylacetyl]amino}methyl)phenyl]methanammonium]₆[V₁₀O₂₈]⁶⁻;
[(3-{[(4-hydroxybenzoyl)amino]methyl}phenyl)methanammonium]₆[V₁₀O₂₈]⁶⁻;
[[3-({[4-(aminomethyl)benzoyl]amino}methyl)phenyl]methanammonium]₆[V₁₀O₂₈]⁶⁻;
[(3-{[(4-bromobenzoyl)amino]methyl}phenyl)methanammonium]₆[V₁₀O₂₈]⁶⁻;
[[3-({[(4-methylphenyl)sulfonyl]amino}methyl)phenyl]methanammonium]₆[V₁₀O₂₈]⁶⁻;
[{3-[(propionylamino)methyl]phenyl}methanammonium]₅[V₁₀O₂₇OH]⁵⁻;
[(3-{[(iodoacetyl)amino]methyl}phenyl)methanammonium]₅[V₁₀O₂₇OH]⁵⁻;
[[3-({[(allyloxy)carbonyl]amino}methyl)phenyl]methanammonium]₅[V₁₀O₂₇OH]⁵⁻;
[{3-[(benzoylamino)methyl]phenyl}methanammonium]₅[V₁₀O₂₇OH]⁵⁻;
[[3-({[(2S)-2-(acetylamino)-2-phenylacetyl]amino}methyl)phenyl]methanammonium]₅[V₁₀O₂₇OH]⁵⁻;
[[3-({[(2R)-2-(acetylamino)-2-phenylacetyl]amino}methyl)phenyl]methanammonium]₅[V₁₀O₂₇OH]⁵⁻;
[(3-{[(4-hydroxybenzoyl)amino]methyl}phenyl)methanammonium]₅[V₁₀O₂₇OH]⁵⁻;
[[3-({[4-(aminomethyl)benzoyl]amino}methyl)phenyl]methanammonium]₅[V₁₀O₂₇OH]⁵⁻;
[(3-{[(4-bromobenzoyl)amino]methyl}phenyl)methanammonium]₅[V₁₀O₂₇OH]⁵⁻;
[[3-({[(4-methylphenyl)sulfonyl]amino}methyl)phenyl]methanammonium]₅[V₁₀O₂₇OH]⁵⁻;
[{3-[(propionylamino)methyl]phenyl}methanammonium]₄[V₁₀O₂₆(OH)₂]⁴⁻;
[(3-{[(iodoacetyl)amino]methyl}phenyl)methanammonium]₄[V₁₀O₂₆(OH)₂]⁴⁻;
[[3-({[(allyloxy)carbonyl]amino}methyl)phenyl]methanammonium]₄[V₁₀O₂₆(OH)₂]⁴⁻;
[{3-[(benzoylamino)methyl]phenyl}methanammonium]₄[V₁₀O₂₆(OH)₂]⁴⁻;
[[3-({[(2S)-2-(acetylamino)-2-phenylacetyl]amino}methyl)phenyl]methanammonium]₄[V₁₀O₂₆(OH)₂]⁴⁻;
[[3-({[(2R)-2-(acetylamino)-2-phenylacetyl]amino}methyl)phenyl]methanammonium]₄[V₁₀O₂₆(OH)₂]⁴⁻;
[(3-{[(4-hydroxybenzoyl)amino]methyl}phenyl)methanammonium]₄[V₁₀O₂₆(OH)₂]⁴⁻;
[[3-({[4-(aminomethyl)benzoyl]amino}methyl)phenyl]methanammonium]₄[V₁₀O₂₆(OH)₂]⁴⁻;
[(3-{[(4-bromobenzoyl)amino]methyl}phenyl)methanammonium]₄[V₁₀O₂₆(OH)₂]⁴⁻;
[[3-({[(4-methylphenyl)sulfonyl]amino}methyl)phenyl]methanammonium]₄[V₁₀O₂₆(OH)₂]⁴⁻;
or a pharmaceutically acceptable salt, solvate, or hydrate thereof.

12. A pharmaceutical composition comprising a compound according to Formula (I), or a pharmaceutically-acceptable salt, solvate, or hydrate thereof, and at least one pharmaceutically-acceptable excipient, diluent or adjuvant thereof.

13. A method of treating type I diabetes in a human comprising administering to the human in need of such treatment a therapeutically effective amount of a compound of Formula (I) or a pharmaceutically-acceptable salt, solvate, or hydrate thereof.

14. A method of treating type II diabetes in a human comprising administering to the human in need of such treatment a therapeutically effective amount of a compound of Formula (I) or a pharmaceutically-acceptable salt, solvate, or hydrate thereof.

15. A method of treating elevated plasma glucose levels in a human comprising administering to the human in need of such treatment a therapeutically effective amount of a compound of Formula (I) or a pharmaceutically-acceptable salt, solvate, or hydrate thereof.

16. A method of treating ketoacidosis in a human comprising administering to the human in need of such treatment a therapeutically effective amount of a compound of Formula (I) or a pharmaceutically-acceptable salt, solvate, or hydrate thereof.

17. A compound of formula: 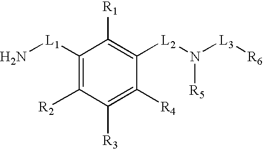
or a pharmaceutically acceptable salt thereof, wherein
L₁ and L₂ are independently (C₁-C₆)alkylene;
L₃ is —C(O)— or —S(O)₂—;
R₁, R₂, R₃, and R₄ are independently H, (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, or nitro;
R₅ is H or (C₁-C₆)alkyl;
R₆ is (C₁-C₆)alkoxy, (C₂-C₆)alkenyl, (C₂-C₆)alkenyloxy, (C₁-C₆)alkyl, (C₂-C₆)alkynyl, (C₂-C₆)alkynyloxy, aryl, (C₁-C₆)haloalkyl, (C₁-C₆)haloalkoxy, NR₇R₈, —CH(R₉)NR₁₀R₁₁, or —CH₂CH₂NR₁₀R₁₁, wherein the aryl is optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl;
R₇ and R₈ are independently H or (C₁-C₆)alkyl;
R₉ is H, (C₁-C₆)alkoxy(C₁-C₆)alkyl, (C₁-C₆)alkoxycarbonyl(C₁-C₆)alkyl, (C₁-C₆)alkyl, thio(C₁-C₆)alkyl, (C₁-C₆)alkylthio(C₁-C₆)alkyl, aryl, aryl(C₁-C₆)alkyl, carboxy(C₁-C₆)alkyl, (C₃-C₇)cycloalkyl, (C₃-C₇)cycloalkyl(C₁-C₆)alkyl, heteroaryl, heteroaryl(C₁-C₆)alkyl, hydroxy(C₁-C₆)alkyl, NH₂C(═NH)NH(C₁-C₆)alkyl, NR₇R₈(C₁-C₆)alkyl, or NR₇R₈carbonyl(C₁-C₆)alkyl, wherein the aryl, heteroaryl, and (C₃-C₇)cycloalkyl are optionally substituted with 1, 2, 3, 4, or 5 substituents independently selected from (C₂-C₆)alkenyl, (C₁-C₆)alkoxy, (C₁-C₆)alkoxycarbonyl, (C₁-C₆)alkyl, (C₁-C₆)alkylcarbonyl, (C₁-C₆)alkylcarbonyloxy, (C₁-C₆)alkylthio, (C₂-C₆)alkynyl, carboxy, cyano, (C₁-C₄)haloalkoxy, (C₁-C₄)haloalkyl, halogen, hydroxy, hydroxy(C₁-C₆)alkyl, mercapto, nitro, oxo, NR₇R₈, and NR₇R₈(C₁-C₆)alkyl; and
R₁₀ and R₁₁ are independently H, (C₁-C₆)alkyl, (C₁-C₆)alkylcarbonyl, formyl, or (C₁-C₆)alkoxycarbonyl;
with the proviso that the formula does not encompass N-(3-(aminomethyl)benzyl)acetamide.

18. The compound according to claim 17 wherein
L₁ is —CH₂—;
L₂ is —CH₂—;
L₃ is —C(O)—;
R₁, R₂, R₃, R₄, and R₅ are H;
R₆ is (C₂-C₆)alkenyloxy, (C₂-C₆)alkyl, (C₁-C₆)haloalkyl or aryl, wherein the aryl is phenyl optionally substituted with 1 substituent selected from halogen, hydroxy, and NR₇R₈(C₁-C₆)alkyl; and
R₇ and R₈ are H.

19. The compound according to claim 17 wherein
L₁ is —CH₂—;
L₂ is —CH₂—;
L₃ is —C(O)—;
R₁, R₂, R₃, R₄, and R₅ are H; and
R₆ is (C₂-C₆)alkenyloxy.

20. The compound according to claim 17 wherein
L₁ is —CH₂—;
L₂ is —CH₂—;
L₃ is —C(O)—;
R₁, R₂, R₃, R₄, and R₅ are H; and
R₆ is (C₂-C₆)alkyl.

21. The compound according to claim 17 wherein
L₁ is —CH₂—;
L₂ is —CH₂—;
L₃ is —C(O)—;
R₁, R₂, R₃, R₄, and R₅ are H; and
R₆ is (C₁-C₆)haloalkyl.

22. The compound according to claim 17 wherein
L₁ is —CH₂—;
L₂ is —CH₂—;
L₃ is —C(O)—;
R₁, R₂, R₃, R₄, and R₅ are H;
R₆ is phenyl optionally substituted with 1 substituent selected from halogen, hydroxy, and —CH₂NH₂.

23. The compound according to claim 17 wherein
L₁ is —CH₂—;
L₂ is —CH₂—;
L₃ is —C(O)—;
R₁, R₂, R₃, R₄, and R₅ are H;
R₆ is —CH(R₉)NR₁₀R₁₁;
R₁₀ is —H; and
R₁₁ is (C₁-C₆)alkylcarbonyl.

24. The compound according to claim 17 wherein
L₁ is —CH₂—;
L₂ is —CH₂—;
L₃ is —C(O)—;
R₁, R₂, R₃, R₄, and R₅ are H;
R₆ is —CH(R₉)NR₁₀R₁₁;
R₉ is phenyl;
R₁₀ is —H; and
R₁₁ is (C₁-C₆)alkylcarbonyl.

25. The compound according to claim 17 wherein
L₁ is —CH₂—;
L₂ is —CH₂—;
L₃ is —S(O)₂—;
R₆ is phenyl optionally substituted with 1 substituent selected from halogen, hydroxy, and —CH₂NH₂.

26. The compound according to claim 17 that is
N-(3-(aminomethyl)benzyl)propionamide;
N-(3-(aminomethyl)benzyl)-2-iodoacetamide;
allyl 3-(aminomethyl)benzylcarbamate;
N-(3-(aminomethyl)benzyl)benzamide;
(S)-2-acetamido-N-(3-(aminomethyl)benzyl)-2-phenylacetamide;
N-(3-(aminomethyl)benzyl)-4-methylbenzenesulfonamide;
N-(3-(aminomethyl)benzyl)-4-hydroxybenzamide;
4-(aminomethyl)-N-(3-(aminomethyl)benzyl)benzamide; or
N-(3-(aminomethyl)benzyl)-4-bromobenzamide;
or a pharmaceutically acceptable salt thereof.

27. A method of treating a disorder ameliorated by the inhibition SSAO/VAP-1 in human comprising administering to the human in need of such treatment a therapeutically effective amount of a compound of claim 17.

28. The method according to claim 27 wherein the disorder is diabetes.

29. A pharmaceutical composition comprising a compound according to claim 17 and at least one pharmaceutically-acceptable excipient, diluent or adjuvant thereof.

30. A method of preparing a compound of formula 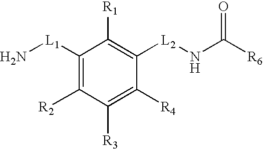
wherein L₁, L₂, R₁, R₂, R₃, R₄, and R₆ are as defined in claim 1, comprising:
(a) treating a hydroxy (—OH) containing resin with 4-nitrophenyl chloroformate
and a base to provide a compound of formula 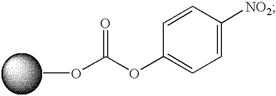
(b) treating the product of (b) with a compound of formula 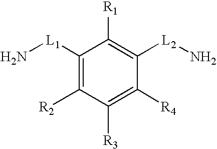
to provide a compound of formula 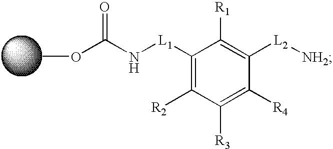
(c) treating the product of (b) with HOBt, DIPCDI, a base, and a compound of
formula R₆C(O)OH to provide a compound of formula 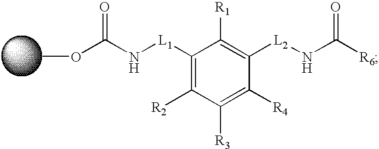
(d) treating the product of (c) with an acid to provide a compound of formula

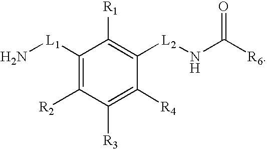

31. A method of preparing a compound of formula 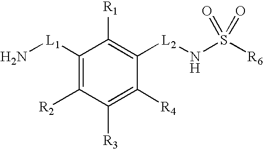
wherein L₁, L₂, R₁, R₂, R₃, R₄, and R₆ are as defined in claim 1, comprising:
(a) treating a hydroxy (—OH) containing resin with 4-nitrophenyl chloroformate
and a base to provide a compound of formula 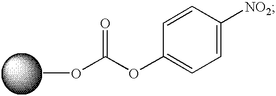
(b) treating the product of (b) with a compound of formula 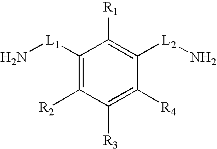
to provide a compound of formula 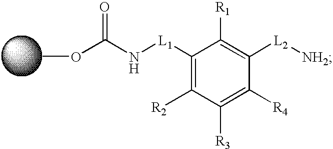
(c) treating the product of (b) with a base and a compound of formula ClS(O)₂R to provide a compound of formula 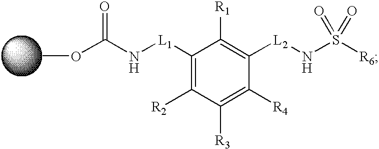
(d) treating the product of (c) with an acid to provide a compound of formula

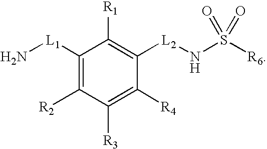

32. A pharmaceutical composition comprising a vanadium salt of a compound according to claim 17 and at least one pharmaceutically-acceptable excipient, diluent or adjuvant thereof.
